(19) United States
(12) Patent Application Publication
(10) Pub. No.: US 2025/0297030 A1
(43) Pub. Date: Sep. 25, 2025

(54) ANTI-MUC17 NANOBODY AND USE THEREOF

(71) Applicant: SHANDONG SIMCERE BIOPHARMACEUTICAL CO., LTD., Yantai, Shandong (CN)

(72) Inventors: Guangcun CHENG, Shanghai (CN); Ling LI, Shanghai (CN); Yayuan FU, Shanghai (CN); Zhuoxiao CAO, Shanghai (CN); Renhong TANG, Shanghai (CN); Jinsheng REN, Nanjing, Jiangsu (CN)

(21) Appl. No.: 18/703,322

(22) PCT Filed: Oct. 20, 2022
(86) PCT No.: PCT/CN2022/126410

§ 371 Date: Apr. 19, 2024

(30) Foreign Application Priority Data
Oct. 21, 2021 (CN) 202111224424.2

(51) Int. Cl. C07K 16/30; A61K 40/31; A61K 40/42; G01N 33/68

(52) U.S. Cl. CPC C07K 16/3092; A61K 40/31; A61K 40/4257; G01N 33/6854; A61K 2239/13; A61K 2239/21; A61K 2239/22; C07K 2317/24; C07K 2317/31; C07K 2317/526; C07K 2317/565; C07K 2317/567; C07K 2317/569; C07K 2317/92; G01N 2333/4725

(57) ABSTRACT

An antibody or an antigen-binding fragment thereof is capable of specifically binding to MUC17. The antibody or the antigen-binding fragment thereof can specifically bind to MUC17 with high affinity and can be used as a drug for treating malignant gastrointestinal tumors.

### CROSS-REFERENCE TO RELATED APPLICATIONS

The present application is the U.S. National Stage Application under 35 U.S.C. § 371 of International Application No. PCT/CN2022/126410, filed Oct. 20, 2022, designating the U.S. and published as WO 2023/066336 A1 on Apr. 27, 2023, which claims priority to the Chinese Patent Application No. 202111224424.2 filed on Oct. 21, 2021, which is incorporated herein by reference in its entirety. Any and all applications for which a foreign or a domestic priority is claimed is/are identified in the Application Data Sheet filed herewith and is/are hereby incorporated by reference in their entirety under 37 C.F.R. § 1.57.

### SEQUENCE LISTING IN ELECTRONIC FORMAT

The present application is being filed along with an Electronic Sequence Listing, which is provided as a file entitled INSG032001APCSEQLIST.XML, created and last saved on Apr. 18, 2024, which is 93,139 bytes in size, which is replaced by a Replacement Electronic Sequence Listing submitted herewith as a file entitled 24C80484US.xml, created on May 8, 2025, which is 93,283 bytes in size. The information in the Electronic Sequence Listing is incorporated herein by reference in its entirety in accordance with 35 U.S.C. § 1.52(e).

### TECHNICAL FIELD

The present application relates to the field of antibodies, in particular to an anti-MUC17 nanobody.

### BACKGROUND

A special antibody lacking a light chain exists in animals of the Camelidae family and cartilaginous fish bodies. A variable region fragment derived from the antibody is generally called a nanobody. The nanobody generally has a long CDR3, can form a stable large convex ring structure, can bind to some hidden epitopes, and thus is particularly suitable for targets where antibodies are difficult to obtain, such as GPCRs, ion channels and enzyme activity centers, etc. Compared with traditional antibodies, the VHH nanobody has a small molecular weight, easy in-vitro expression, good solubility, and weak immunogenicity, and can penetrate through some protective barriers in a body to enter the diseased region to play a role, such as a blood-brain barrier.

Mucins are a family of high molecular weight glycoproteins that can be classified as secreted proteins and transmembrane proteins, whose serine- and threonine-rich protein skeletons are bound to a variety of O-type oligosaccharide side chains. Typical oligosaccharide chains account for about 70% of mature mucins. Membrane-binding mucins, i.e., cell surface mucins, bind to cell membranes by means of transmembrane regions. The members include MUC1, MUC3A, MUC3B, MUC4, MUC12, MUC13, MUC15, MUC16, and MUC17. MUC17 (Mucin-17) is a single transmembrane mucin and has high homology with MUC3, which was found for the first time in 2002. MUC17 protein has a total length of 4493 amino acids. The extracellular segment comprises 59 tandem repeats rich in serine, threonine, and proline, 2 EGF domains, and 1 SEA domain. The intracellular segment comprises 80 amino acid cytoplasmic tails and phosphorylation sites, wherein phosphorylation modification of the intracellular segment may be related to signaling of cell proliferation and apoptosis. Different types of membrane-binding mucins are expressed at different sites. For example, in a normal human body, MUC1 is mainly expressed in the stomach, and MUC17 is mainly expressed in the intestinal tract. In tumor tissues, MUC17 is highly expressed in both gastric cancer and pancreatic cancer, and belongs to tumor-associated antigens. The development of drugs targeting MUC17 is of great significance for clinical treatment of gastric cancer and pancreatic cancer. Currently, for the MUC17 target, only the Amgen bispecific antibody AMG199 was clinically approved for the treatment of MUC17 positive gastric cancer or gastroesophageal junction carcinoma, which produces a cytotoxic effect against tumor cells expressing MUC17 by simultaneously binding to CD3 on T cells and MUC17 on tumor cells.

### SUMMARY

The MUC17 specific nanobody obtained by screening in the present application is intended to be used for treating different forms of malignant tumors. The present application provides an anti-MUC17 antibody, a nucleic acid for encoding the antibody, an antibody preparation method, a pharmaceutical composition comprising the antibody, and related use of the pharmaceutical composition in treating a tumor.

In a first aspect, the present application provides a nanobody or an antigen-binding fragment thereof specifically binding to MUC17, wherein the nanobody or the antigen-binding fragment thereof comprises a CDR1, a CDR2, and a CDR3; the CDR1, the CDR2, and the CDR3 are respectively selected from a CDR1, a CDR2, and a CDR3 of the VHH set forth in any one of SEQ ID NOs: 8-12, 62-66, and 67-98.

In a second aspect, the present application provides a multispecific molecule, wherein the multispecific molecule comprises the nanobody or the antigen-binding fragment thereof according to the first aspect.

In a third aspect, the present application provides a chimeric antigen receptor (CAR), wherein the chimeric antigen receptor at least comprises an extracellular antigen-binding domain, a transmembrane domain, and an intracellular signaling domain; the extracellular antigen-binding domain comprises the nanobody or the antigen-binding fragment thereof according to the first aspect.

In a fourth aspect, the present application provides an immune effector cell, wherein the immune effector cell expresses the chimeric antigen receptor according to the third aspect or comprises a nucleic acid fragment encoding the chimeric antigen receptor according to the third aspect.

In a fifth aspect, the present application provides an isolated nucleic acid fragment, wherein the nucleic acid fragment encodes the nanobody or the antigen-binding fragment thereof according to the first aspect, or the multispecific molecule according to the second aspect, or the chimeric antigen receptor according to the third aspect.

In a sixth aspect, the present application provides a vector, wherein the vector comprises the nucleic acid fragment according to the fifth aspect.

In a seventh aspect, the present application provides a host cell, wherein the host cell comprises the vector according to the sixth aspect.

In an eighth aspect, the present application provides a method for preparing the nanobody or the antigen-binding fragment thereof according to the first aspect or the multispecific molecule according to the second aspect, wherein the method comprises culturing the cell according to the seventh aspect, and isolating the nanobody or the antigen-binding fragment thereof expressed by the cell, or isolating a multispecific molecule expressed by the cell.

In a ninth aspect, the present application provides a method for preparing the immune effector cell according to the fourth aspect, wherein the method comprises introducing a nucleic acid fragment encoding the CAR according to the third aspect into the immune effector cell.

In a tenth aspect, the present application provides a pharmaceutical composition, wherein the pharmaceutical composition comprises the nanobody or the antigen-binding fragment thereof according to the first aspect, the multispecific antibody according to the second aspect, the immune effector cell according to the fourth aspect, the nucleic acid fragment according to the fifth aspect, the vector according to the sixth aspect, the host cell according to the seventh aspect, or a product prepared by the method according to the eighth aspect.

In an eleventh aspect, the present application provides use of the nanobody or the antigen-binding fragment thereof according to the first aspect, the multispecific antibody according to the second aspect, the immune effector cell according to the fourth aspect, the nucleic acid fragment according to the fifth aspect, the vector according to the sixth aspect, the host cell according to the seventh aspect, a product prepared by the method according to the eighth aspect, or the pharmaceutical composition according to the tenth aspect in preparing a medicament for preventing and/or treating a tumor.

In a twelfth aspect, the present application provides a method for preventing and/or treating a tumor, comprising administering to a patient in need thereof an effective amount of the nanobody or the antigen-binding fragment thereof according to the first aspect, the multispecific antibody according to the second aspect, the immune effector cell according to the fourth aspect, the nucleic acid fragment according to the fifth aspect, the vector according to the sixth aspect, the host cell according to the seventh aspect, a product prepared by the method according to the eighth aspect, or the pharmaceutical composition according to the tenth aspect.

In a thirteenth aspect, the present application provides use of the nanobody or the antigen-binding fragment thereof according to the first aspect, the multispecific antibody according to the second aspect, the immune effector cell according to the fourth aspect, the nucleic acid fragment according to the fifth aspect, the vector according to the sixth aspect, the host cell according to the seventh aspect, a product prepared by the method according to the eighth aspect, or the pharmaceutical composition according to the tenth aspect in preventing and/or treating a tumor.

In a fourteenth aspect, the present application provides a kit, wherein the kit comprises the nanobody or the antigen-binding fragment thereof according to the first aspect, the multispecific antibody according to the second aspect, the immune effector cell according to the fourth aspect, the nucleic acid fragment according to the fifth aspect, the vector according to the sixth aspect, the host cell according to the seventh aspect, a product prepared by the method according to the eighth aspect, or the pharmaceutical composition according to the tenth aspect.

In a fifteenth aspect, the present application provides a method for detecting MUC17 expression, comprising contacting a sample to be tested with the nanobody or the antigen-binding fragment thereof according to the first aspect in a condition allowing formation of a complex by the nanobody or the antigen-binding fragment thereof according to the first aspect and MUC17.

In a sixteenth aspect, the present application provides a method for inhibiting the proliferation or migration of a cell expressing MUC17 in vitro, comprising contacting the cell with the nanobody or the antigen-binding fragment thereof according to the first aspect in a condition allowing formation of a complex by the nanobody or the antigen-binding fragment thereof according to the first aspect and MUC17.

### BRIEF DESCRIPTION OF THE DRAWINGS

FIG. 1A shows binding reactions of Lab315-huFc recombinant antibodies with a human MUC17 ECD4131-his protein by ELISA.

FIG. 1B shows binding reactions of Lab316-huFc, Lab320-huFc, Lab331-huFc, and Lab332-huFc recombinant antibodies with a human MUC17 ECD4131-his protein by ELISA.

FIG. 2A shows binding reactions of Lab315-huFc recombinant antibodies with a monkey MUC17 ECD3577-His protein by ELISA.

FIG. 2B shows binding reactions of Lab316-huFc, Lab320-huFc, Lab331-huFc, and Lab332-huFc recombinant antibodies with a monkey MUC17 ECD3577-His protein by ELISA.

FIG. 3A shows binding reactions of Lab315-huFc recombinant antibodies with NUGC4 endogenous tumor cells by FACS.

FIG. 3B shows binding reactions of Lab316-huFc, Lab320-huFc, Lab331-huFc, and Lab332-huFc recombinant antibodies with NUGC4 endogenous tumor cells by FACS.

FIG. 4A shows binding of Lab315-huFc recombinant antibodies with cells overexpressing monkey MUC17 FlpinCHO-Cyno(3597-3964)-D2 by FACS.

FIG. 4B shows binding of Lab316-huFc, Lab320-huFc, Lab331-huFc, and Lab332-huFc recombinant antibodies with cells overexpressing monkey UC17 FlpinCHO-Cyno(3597-3964)-D2 by FACS.

FIGS. 5A-5E show binding reactions of SCR-7724, SCR-7782, SCR-7783, SCR-7784, and SCR-7785 humanized antibodies with a human MUC17 ECD4131-his protein by ELISA, respectively.

FIGS. 6A-6E show binding reactions of SCR-7724, SCR-7782, SCR-7783, SCR-7784, and SCR-7785 humanized antibodies with a monkey MUC17 ECD3577-His protein by ELISA, respectively.

FIGS. 7A-7E show binding reactions of SCR-7724, SCR-7782, SCR-7783, SCR-7784, and SCR-7785 humanized antibodies with NUGC4 endogenous cells by FACS, respectively.

FIGS. 8A-8E show binding of SCR-7724, SCR-7782, SCR-7783, SCR-7784, and SCR-7785 humanized antibodies with cells overexpressing monkey MUC17 FlpinCHO-Cyno(3597-3964)-D2 by FACS, respectively.

### DETAILED DESCRIPTION

### Terminology and Definitions

Unless otherwise defined herein, scientific and technical terms used in correlation with the present application shall have the meanings that are commonly understood by those skilled in the art.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “including”, “comprising”, and “having” herein are used interchangeably and are intended to indicate the inclusion of a solution, implying that there may be elements other than those listed in the solution. Meanwhile, it should be understood that the descriptions “including”, “comprising”, and “having” as used herein also provide the solution of “consisting of . . . ”. Illustratively, “a composition, comprising A and B” should be understood as the following technical solution: a composition consisting of A and B, and a composition containing other components in addition to A and B, all fall within the scope of the aforementioned “a composition”.

The term “and/or” as used herein includes the meanings of “and”, “or”, and “all or any other combination of elements linked by the term”.

The term “MUC17” as used herein refers to a member of the mucin family. The mucin family includes more than 20 members. Mucins are large and highly glycosylated membrane-binding proteins that are expressed almost exclusively in the intestinal tract. Their general function is to protect epithelial cells from environmental influences, as well as to regulate the proliferation and survival of cells.

MUC17 is highly expressed in pancreatic adenocarcinoma tissues. MUC17 is expressed in pancreatic cancer, appendiceal cancer, and some colon cancers. Its expression is not detected in cell lines in normal pancreas and pancreatitis or derived from other cancers.

The term “specific binding” herein means that an antigen-binding molecule (e.g., an antibody) specifically binds to an antigen and substantially identical antigens, generally with high affinity, but does not bind to unrelated antigens with high affinity. Affinity is generally reflected in an equilibrium dissociation constant (KD), where a relatively low KD indicates a relatively high affinity. In the case of antibodies, high affinity generally means having a KD of 1×10⁻⁷ M or less, about 1×10⁻⁸ M or less, about 1×10⁻⁹ M or less, about 1×10⁻¹⁰ M or less, 1×10⁻¹¹ M or less, or 1×10⁻¹² M or less. KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis. Illustratively, KD can be obtained by the method as described in Example 3 or 6 herein.

The term “antigen-binding molecule” herein is used in its broadest sense and refers to a molecule that specifically binds to an antigen. Illustratively, the antigen-binding molecule includes, but is not limited to, an antibody or an antibody mimetic. “Antibody mimetic” refers to an organic compound or a binding domain that is capable of specifically binding to an antigen, but is not structurally related to an antibody. Illustratively, the antibody mimetic includes, but is not limited to, affibody, affitin, affilin, a designed ankyrin repeat protein (DARPin), a nucleic acid aptamer, and a Kunitz domain peptide.

The term “antibody” herein is used in its broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen-binding activity. “Antibody” herein includes alternative protein scaffolds or artificial scaffolds having grafted complementarity determining regions (CDRs) or CDR derivatives. Such scaffolds include antibody-derived scaffolds comprising mutations introduced to, for example, stabilize the three-dimensional structure of the antibody, and fully synthetic scaffolds comprising, for example, biocompatible polymers. See, e.g., Korndorfer et al., 2003, Proteins: Structure, Function, and Bioinformatics, 53(1): 121-129 (2003); and Roque et al., Biotechnol. Prog. 20: 639-654 (2004). Such scaffolds may also include non-antibody derived scaffolds, such as scaffold proteins known in the art to be useful for grafting CDRs, including, but not limited to tenascin, fibronectin, peptide aptamers, and the like.

“Antibody” herein includes antibodies that do not comprise a light chain, e.g., heavy chain antibodies (HCAbs) produced by Camelidae species such as Camelus dromedarius, Camelus bactrianus, Lama glama, Lama guanicoe, and Vicugna pacos, as well as immunoglobulin new antigen receptors (IgNARs) found in Chondrichthyes, e.g., shark.

As used herein, the term “heavy chain antibody” refers to an antibody lacking a light chain of a conventional antibody. The term specifically includes, but is not limited to, homodimeric antibodies comprising a VH antigen-binding domain and CH2 and CH3 constant domains in the absence of a CH1 domain.

As used herein, the term “nanobody” refers to a heavy chain antibody naturally lacking a light chain present in a camel, and the cloning of its variable region can give a single domain antibody only consisting of a heavy chain variable region (also called VHH (variable domain of heavy chain of heavy chain antibody)), which is the smallest functional antigen-binding fragment.

The terms “nanobody” and “single domain antibody (sdAb)” herein have the same meaning and can be used interchangeably, and refer to a single domain antibody consisting of only one heavy chain variable region constructed by cloning a variable region of a heavy chain antibody, which is the smallest antigen-binding fragment having the complete function. Generally, a single domain antibody consisting of only one heavy chain variable region is constructed by obtaining a heavy chain antibody naturally lacking a light chain and a heavy chain constant region 1 (CH1) and then cloning a variable region of an antibody heavy chain.

For further description of “heavy chain antibody” and “nanobody”, see: Hamers-Casterman et al., Nature. 1993; 363; 446-8; a review article (Reviews in Molecular Biotechnology 74: 277-302, 2001) by Muyldermans; and the following patent applications mentioned as general background art: WO 94/04678, WO 95/04079, and WO 96/34103; WO94/25591, WO 99/37681, WO 00/40968, WO 00/43507, WO 00/65057, WO 01/40310, WO 01/44301, EP 1134231, and WO 02/48193; WO97/49805, WO 01/21817, WO 03/035694, WO 03/054016, and WO 03/055527; WO 03/050531; WO 01/90190; WO03/025020; and WO 04/041867, WO 04/041862, WO 04/041865, WO 04/041863, WO 04/062551, WO 05/044858, WO 06/40153, WO 06/079372, WO 06/122786, WO 06/122787, and WO 06/122825, as well as other prior art mentioned in these applications. “Antibody” herein may be derived from any animal, including, but not limited to, human and non-human animals which may be selected from primates, mammals, rodents, and vertebrates, such as Camelidae species, Lama glama, Lama guanicoe, Vicugna pacos, sheep, rabbits, mice, rats, or Chondrichthyes (e.g., shark).

The term “multispecific” herein means having at least two antigen-binding sites, each of which binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “valent” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule.

“Antigen-binding fragment” and “antibody fragment” herein are used interchangeably and do not have the entire structure of an intact antibody, but comprise only a portion of the intact antibody or a variant of the portion that has the ability to bind to an antigen. “Antigen-binding fragment” or “antibody fragment” herein includes but is not limited to, a Fab, a Fab′, a Fab′-SH, a F(ab′)₂, an Fd, an Fv, an scFv, a diabody, and a single domain antibody.

The term “chimeric antibody” herein refers to an antibody in which a portion of the light chain or/and heavy chain is derived from one antibody (which may be derived from a particular species or belong to a particular antibody class or subclass) and another portion of the light chain or/and heavy chain is derived from another antibody (which may be derived from the same or a different species or belong to the same or a different antibody class or subclass), but which nevertheless retains binding activity to a target antigen (U.S. Pat. No. 4,816,567 to Cabilly et al.; Morrison et al., Proc. Natl. Acad. Sci. USA, 81: 6851 6855(1984)). For example, the term “chimeric antibody” can include an antibody (e.g., a human-murine chimeric antibody) in which the heavy and light chain variable regions of the antibody are derived from a first antibody (e.g., a murine antibody) and the heavy and light chain constant regions of the antibody are derived from a second antibody (e.g., a human antibody).

The term “humanized antibody” herein refers to a genetically engineered non-human antibody that has an amino acid sequence modified to increase homology to the sequence of a human antibody. Generally, all or part of the CDRs of a humanized antibody is derived from a non-human antibody (donor antibody), and all or part of the non-CDRs (e.g., variable region FRs and/or constant regions) is derived from a human immunoglobulin (receptor antibody). The humanized antibody generally retains or partially retains the desired properties of the donor antibody, including, but not limited to, antigen specificity, affinity, reactivity, the ability to increase the activity of immune cells, the ability to enhance immune response, and the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The fully human antibody herein may include amino acid residues that are not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences.

The term “variable region” herein refers to a region of a heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains (VH and VL, respectively) of natural antibodies generally have similar structures, each of which contains four conservative framework regions (FRs) and three hypervariable regions (HVRs). See, e.g., Kindt et al., Kuby Immunology, 6th ed., W. H. Freeman and Co., p. 91 (2007). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region because it is precisely complementary to an epitope in a spatial structure, wherein the heavy chain variable region CDR may be abbreviated as HCDR and the light chain variable region CDR may be abbreviated as LCDR. The terms “framework region” or “FR” are used interchangeably and refer to those amino acid residues of an antibody heavy chain variable region or light chain variable region, other than CDRs. Generally, a typical antibody variable region consists of 4 FRs and 3 CDRs in the following order: FR1-CDR1-FR2-CDR2-FR3-CDR3-FR4.

For further description of the CDRs, see Kabat et al., J. Biol. Chem., 252: 6609-6616 (1977); Kabat et al., United States Department of Health and Human Services, Sequences of proteins of immunological interest (1991); Chothia et al., J. Mol. Biol. 196: 901-917 (1987); Al-Lazikani B. et al., J. Mol. Biol., 273: 927-948 (1997); MacCallum et al., J. Mol. Biol. 262: 732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309: 657-670 (2001). “CDR” herein may be labeled and defined in a manner well known in the art, including, but not limited to, Kabat numbering scheme, Chothia numbering scheme, or IMGT numbering scheme; the tool sites used include, but are not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi#results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “IMGT numbering scheme” herein generally refers to a numbering scheme based on the international ImMunoGeneTics information system (IMGT) initiated by Lefranc et al., see Lefranc et al., Dev. Comparat. Immunol. 27: 55-77, 2003.

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J Mol. Biol. 196: 901-917; Chothia et al., (1989) Nature 342: 878-883).

The term “Fc” herein refers to the carboxyl-terminal portion of an antibody that is formed by the hydrolysis of an intact antibody by papain, which typically comprises the CH3 and CH2 domains of the antibody. The Fc region includes, for example, an Fc region of native sequences, a recombinant Fc region, and a variant Fc region. Although the boundaries of the Fc region of an immunoglobulin heavy chain may vary slightly, the human IgG heavy chain Fc region is generally defined to stretch from an amino acid residue at position Cys226, or from Pro230, to the carboxyl terminus thereof. The C-terminal lysine of the Fc region (residue 447 according to the Kabat numbering scheme) may be removed, for example, during production or purification of the antibody, or by recombinant engineering of the nucleic acid encoding the heavy chain of the antibody, and thus, the Fc region may or may not include Lys447.

The term “conservative amino acid” herein generally refers to amino acids that belong to the same class or have similar characteristics (e.g., charge, side chain size, hydrophobicity, hydrophilicity, backbone conformation, and rigidity). Illustratively, the amino acids in each of the following groups belong to conservative amino acid residues of each other, and substitutions of amino acid residues within the groups belong to conservative amino acid substitutions:

Illustratively, the following six groups are examples of amino acids that are considered to be conservative replacements of each other:
1) alanine (A), serine (S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The term “identity” herein can be obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm ((1970) J. Mol. Biol., 48: 444-453; available at www.gcg.com) which has been integrated into the GAP program of the GCG software package, using the Blossum 62 matrix or PAM250 matrix and gap weight of 16, 14, 12, 10, 8, 6, or 4 and length weight of 1, 2, 3, 4, 5, or 6. For another example, the percent identity between two nucleotide acid sequences is determined with the GAP program of the GCG software package (available at www.gcg.com), using the NWSgapdna.CMP matrix and gap weight of 40, 50, 60, 70, or 80 and length weight of 1, 2, 3, 4, 5, or 6. A particularly preferred parameter set (and one that should be used unless otherwise stated) is a Blossum62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W. Miller algorithm ((1989) CABIOS, 4: 11-17) which has been incorporated into the ALIGN program (version 2.0).

Additionally or alternatively, the nucleic acid sequences and protein sequences described herein can be further used as “query sequences” to perform searches against public databases to, e.g., identify other family member sequences or related sequences. For example, such searches can be performed using the NBLAST and XBLAST programs (version 2.0) of Altschul et al., (1990) J Mol. Biol., 215: 403-10. BLAST nucleotide searches can be performed using the NBLAST program, with a score of 100 and a word length of 12, to obtain nucleotide sequences homologous to the nucleic acid (e.g., nucleic acid encoding SEQ ID NO: 1) molecule of the present application. BLAST protein searches can be performed using the XBLAST program, with a score of 50 and a word length of 3, to obtain amino acid sequences homologous to the protein molecule of the present application. To obtain gapped alignment results for the purpose of comparison, gapped BLAST can be used as described in Altschul et al. (1997) Nucleic Acids Res. 25: 3389-3402. When using the BLAST and gapped BLAST programs, the default parameters of the respective programs (e.g., XBLAST and NBLAST) can be used. See www.ncbi.nlm.nih.gov.

The term “chimeric antigen receptor (CAR)” herein refers to an artificial cell surface receptor engineered to be expressed on an immune effector cell and specifically bound to an antigen, which comprises at least (1) an extracellular antigen-binding domain, e.g., a variable heavy or light chain of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. In this context, the term “nucleic acid molecule” encompasses deoxyribonucleic acid (DNA), including, e.g., complementary DNA (cDNA) and genomic DNA; ribonucleic acid (RNA), in particular messenger RNA (mRNA); the synthetic forms of DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as vectors for direct expression of the antibodies of the present application in vitro and/or in vivo, e.g., in a host or patient. Such DNA (e.g., cDNA) or RNA (e.g., mRNA) vectors may be unmodified or modified. For example, mRNA may be chemically modified to enhance the stability of the RNA vector and/or the expression of the encoded molecule, so that the mRNA can be injected into a subject to produce antibodies in vivo (see, e.g., Stadler et al., Nature Medicine 2017, published online, Jun. 12, 2017, doi: 10.1038/nm.4356 or EP 2 101 823 B1). “Isolated” nucleic acid herein refers to a nucleic acid molecule that has been separated from components of its natural environment. The isolated nucleic acid includes a nucleic acid molecule contained in a cell that generally contains the nucleic acid molecule, but the nucleic acid molecule is present extrachromosomally or at a chromosomal location different from its natural chromosomal location.

The term “vector” herein refers to a nucleic acid molecule capable of amplifying another nucleic acid to which it has been linked. The term includes vectors that serve as self-replicating nucleic acid structures as well as vectors integrated into the genome of a host cell into which they have been introduced. Certain vectors are capable of directing the expression of nucleic acids to which they are operably linked. Such vectors are called “expression vectors” herein.

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective, and does not contain additional ingredients having unacceptable toxicity to a subject to which the pharmaceutical composition is administered.

The term “treatment” herein refers to surgical or therapeutic treatment for the purpose of preventing or slowing (reducing) the progression of an undesired physiological or pathological change, e.g., a cancer, in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission (whether partial or total), whether detectable or undetectable. Subjects in need of treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

The term “subject” herein refers to an organism that receives treatment for a particular disease or disorder described herein. Examples of subjects and patients include mammals, such as humans, primates (e.g., monkey), or non-primate mammals, that receive treatment for a disease or disorder.

The term “effective amount” herein refers to an amount of a therapeutic agent that is effective to prevent or alleviate symptoms of a disease or the progression of the disease when administered to a cell, tissue, or subject alone or in combination with another therapeutic agent. “Effective amount” also refers to an amount of a compound that is sufficient to alleviate symptoms, e.g., to treat, cure, prevent, or alleviate related medical disorders, or to increase the rates at which such disorders are treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially, or simultaneously.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

The term “EC50” herein refers to the half maximum effective concentration, which includes the antibody concentration that induces a halfway response between the baseline and maximum after a specified exposure time. EC50 essentially represents the antibody concentration at which 50% of the maximal effect is observed, and can be measured by methods known in the art.

### DETAILED DESCRIPTION

Various nanobodies specifically binding to MUC17 are obtained by screening in the present application, and various humanized forms of nanobodies are obtained by utilizing the nanobodies.

The nanobodies have application prospect in treating MUC17-expressing tumors.

Specifically, the present application provides the following technical solutions:

In a first aspect, the present application provides a nanobody or an antigen-binding fragment thereof specifically binding to MUC17, wherein the antibody or the antigen-binding fragment comprises a CDR1, a CDR2, and a CDR3; the CDR1, the CDR2, and the CDR3 are respectively selected from a CDR1, a CDR2, and a CDR3 of the VHH set forth in any one of SEQ ID NOs: 8-12, 62-66, and 67-98.

In some embodiments, the CDR1, the CDR2, and the CDR3 are determined according to the Kabat numbering scheme, the Chothia numbering scheme, or the IMGT numbering scheme. For example, the CDR1 comprises the amino acid sequence set forth in SEQ ID NO: 13, 16, 19, 22, 25, 28, 31, 34, 37, 40, 43, 46, 49, 52, or 55; the CDR2 comprises the amino acid sequence set forth in SEQ ID NO: 14, 17, 20, 23, 26, 29, 32, 35, 38, 41, 44, 47, 50, 53, or 56; the CDR3 comprises the amino acid sequence set forth in SEQ ID NO: 15, 18, 21, 24, 27, 30, 33, 36, 39, 42, 45, 48, 51, 54, or 57.

In some embodiments, CDR1s, CDR2s, and CDR3s of the VHHs set forth in SEQ ID NOs: 8, 62, and 67-71 are according to the IMGT, Kabat, or Chothia numbering scheme, and have the amino acid sequences set forth in SEQ ID NOs: 13-15, SEQ ID NOs: 28-30, or SEQ ID NOs: 43-45;
CDR1-3s of the VHHs set forth in SEQ ID NOs: 9, 63, and 72-77 are according to the IMGT, Kabat, or Chothia numbering scheme, and have the amino acid sequences set forth in SEQ ID NOs: 16-18, SEQ ID NOs: 31-33, or SEQ ID NOs: 46-48;
CDR1-3s of the VHHs set forth in SEQ ID NOs: 10, 64, and 78-84 are according to the IMGT, Kabat, or Chothia numbering scheme, and have the amino acid sequences set forth in SEQ ID NOs: 19-21, SEQ ID NOs: 34-36, or SEQ ID NOs: 49-51;
CDR1-3s of the VHHs set forth in SEQ ID NOs: 11, 65, and 85-91 are according to the IMGT, Kabat, or Chothia numbering scheme, and have the amino acid sequences set forth in SEQ ID NOs: 22-24, SEQ ID NOs: 37-39, or SEQ ID NOs: 52-54;
CDR1-3s of the VHHs set forth in SEQ ID NOs: 12, 66, and 92-98 are according to the IMGT, Kabat, or Chothia numbering scheme, and have the amino acid sequences set forth in SEQ ID NOs: 25-27, SEQ ID NOs: 40-42, or SEQ ID NOs: 55-57.

In some embodiments, the nanobody or the antigen-binding fragment thereof comprises CDR1, CDR2, and CDR3 sequences having at least 80%, 85%, 90%, 91%, 92%, 93%, 94%, 95%, 96%, 97%, 98%, 99%, or 100% identity to the CDR1, the CDR2, and the CDR3 or having 1, 2, 3, or more amino acid insertions, deletions, and/or substitutions compared with the CDR1, the CDR2, and the CDR3, and preferably, the substitutions are conservative amino acid substitutions.

In some embodiments, the nanobody or the antigen-binding fragment thereof comprises the VHH set forth in any one of SEQ ID NOs: 8-12, 62-66, and 67-98, or a VHH sequence having at least 80%, 85%, 90%, 91%, 92%, 93%, 94%, 95%, 96%, 97%, 98%, 99%, or 100% identity to the VHH set forth in any one of SEQ ID NOs: 8-12, 62-66, and 67-98 or at most 20, 19, 18, 17, 16, 15, 14, 13, 12, 11, 10, 9, 8, 7, 6, 5, 4, 3, 2, or 1 mutation compared with the VHH set forth in any one of SEQ ID NOs: 8-12, 62-66, and 67-98; the mutation may be selected from an insertion, a deletion, and/or a substitution, and preferably, the substitution is a conservative amino acid substitution.

In some embodiments, the nanobody or the antigen-binding fragment thereof comprises a framework region sequence at least having a mutation, compared with a framework region of the VHH set forth in SEQ ID NO: 62, selected from the group consisting of: numbered in the natural order, H34G, V36F, G43E, L44R, W46G, S48A, G57I, R70K, Y93S, W114R, or M119Q; preferably, at least having H34G, V36F, G43E, L44R, W46G, S48A, G57I, R70K, Y93S, and W114R mutations; more preferably, at least having H34G, V36F, G43E, L44R, W46G, S48A, and Wi14R mutations; more preferably, at least having H34G, V36F, G43E, L44R, W46G, S48A, Y93S, and W114R mutations; more preferably, at least having H34G, V36F, G43E, L44R, W46G, S48A, R70K, Y93S, and W114R mutations; more preferably, at least having H34G, V36F, G43E, L44R, W46G, S48A, R70K, Y93S, W114R, and M119Q mutations;
The nanobody or the antigen-binding fragment thereof comprises a framework region sequence at least having a mutation, compared with a framework region of the VHH set forth in SEQ ID NO: 63, selected from the group consisting of: numbered in the natural order, E1D, V5Q, E6A, G26D, F27N, V37F, G44E, L45R, W47G, R71Q, V92M, R97A, or M118Q; preferably, at least having V37F, G44E, L45R, W47G, and R97A mutations; more preferably, at least having V37F, G44E, L45R, W47G, R71Q, and R97 mutations; more preferably, at least having F27N, V37F, G44E, L45R, W47G, R71Q, and R97A mutations; more preferably, at least having E1D, G26D, F27N, V37F, G44E, L45R, W47G, R71Q, and R97A mutations; more preferably, at least having E1D, V5Q, E6A, G26D, F27N, V37F, G44E, L45R, W47G, R71Q, and R97A mutations; more preferably, at least having E1D, G26D, F27N, V37F, G44E, L45R, W47G, R71Q, V92M, R97A, and M118Q mutations;
The nanobody or the antigen-binding fragment thereof comprises a framework region sequence at least having a mutation, compared with a framework region of the VHH set forth in SEQ ID NO: 64, selected from the group consisting of: numbered in the natural order, E1D, V5Q, E6A, T28P, F29S, V37F, G44E, L45R, W47G, K75L, N76S, Y79W, V92M, R97A, or M118Q; preferably, at least having V37F, G44E, L45R, W47G, and R97A mutations; more preferably, at least having T28P, F29S, V37F, G44E, L45R, W47G, and R97A mutations; more preferably, at least having E1D, T28P, F29S, V37F, G44E, L45R, W47G, and R97A mutations; more preferably, at least having E1D, T28P, F29S, V37F, G44E, L45R, W47G, K75L, N76S, and R97A mutations; more preferably, at least having E1D, T28P, F29S, V37F, G44E, L45R, W47G, K75L, N76S, Y79W, and R97A mutations; more preferably, at least having E1D, V5Q, E6A, T28P, F29S, V37F, G44E, L45R, W47G, K75L, N76S, and R97A mutations; more preferably, at least having E1D, T28P, F29S, V37F, G44E, L45R, W47G, K75L, N76S, V92M, R97A, and M118Q mutations;
The nanobody or the antigen-binding fragment thereof comprises a framework region sequence at least having a mutation, compared with a framework region of the VHH set forth in SEQ ID NO: 65, selected from the group consisting of: numbered in the natural order, E1D, V5Q, E6A, H35A, V37F, G44E, L45R, W47V, F68L, R72H, S75A, V93M, or M119Q; preferably, at least having H35A, V37F, G44E, L45R, and W47V mutations; more preferably, at least having H35A, V37F, G44E, L45R, W47V, and R72H mutations; more preferably, at least having E1D, H35A, V37F, G44E, L45R, W47V, and R72H mutations; more preferably, at least having E1D, H35A, V37F, G44E, L45R, W47V, R72H, and S75A mutations; more preferably, at least having E1D, H35A, V37F, G44E, L45R, W47V, F68L, R72H, and S75A mutations; more preferably, at least having E1D, V5Q, E6A, H35A, V37F, G44E, L45R, W47V, and R72H mutations; more preferably, at least having E1D, H35A, V37F, G44E, L45R, W47V, R72H, V93M, and M119Q mutations;
or the nanobody or the antigen-binding fragment comprises a framework region sequence at least having a mutation, compared with a framework region of the VHH set forth in SEQ ID NO: 66, selected from the group consisting of: numbered in the natural order, V5Q, E6A, F27D, V37F, A40R, G44E, L45R, W47A, R71Q, S74T, V92M, K97A, or M118Q; preferably, at least having V37F, G44E, L45R, W47A, and K97A mutations; more preferably, at least having V37F, G44E, L45R, W47A, R71Q, and K97A mutations; more preferably, at least having F27D, V37F, G44E, L45R, W47A, R71Q, and K97A mutations; more preferably, at least having F27D, V37F, G44E, L45R, W47A, R71Q, S74T, and K97A mutations; more preferably, at least having F27D, V37F, G44E, L45R, W47A, R71Q, V92M, K97A, and M118Q mutations; more preferably, at least having F27D, V37F, A40R, G44E, L45R, W47A, R71Q, V92M, K97A, and M118Q mutations; more preferably, at least having V5Q, E6A, F27D, V37F, G44E, L45R, W47A, R71Q, and K97A mutations.

In some embodiments, the nanobody or the antigen-binding fragment thereof specifically binds to a human MUC17 protein and/or a monkey MUC17 protein; preferably, the nanobody or the antigen-binding fragment thereof binds to human MUC17 protein and/or monkey MUC17 protein with a KD superior to 1.00E−7 M.

In some embodiments, the nanobody or the antigen-binding fragment thereof is: (1) a chimeric antibody or a fragment thereof, (2) a humanized antibody or a fragment thereof, or (3) a fully human antibody or a fragment thereof.

In some embodiments, the nanobody or the antigen-binding fragment thereof comprises or does not comprise an antibody heavy chain constant region; optionally, the antibody heavy chain constant region may be selected from human, Vicugna pacos, mouse, rat, rabbit, or sheep; optionally, the antibody heavy chain constant region may be selected from IgG, IgM, IgA, IgE, or IgD, and the IgG may be selected from IgG1, IgG2, IgG3, or IgG4; optionally, the heavy chain constant region may be selected from an Fc region, a CH3 region, or an intact heavy chain constant region; preferably, the heavy chain constant region is a human Fc region; preferably, the nanobody or the antigen-binding fragment thereof is a heavy chain antibody.

In some embodiments, the nanobody or the antigen-binding fragment thereof is further conjugated to a therapeutic agent or a tracer; preferably, the therapeutic agent is selected from a drug, a toxin, a radioisotope, a chemotherapeutic agent, or an immunomodulator, and the tracer is selected from a radiocontrast medium, a paramagnetic ion, a metal, a fluorescent label, a chemiluminescent label, an ultrasound contrast agent, and a photosensitizer.

In another aspect, the present application provides a multispecific molecule comprising any one of the foregoing nanobodies or antigen-binding fragment thereof, preferably, the multispecific molecule further comprises a nanobody or an antigen-binding fragment thereof specifically binding to an antigen other than MUC17 or binding to a MUC17 epitope different from that of any one of the foregoing nanobodies or the antigen-binding fragment thereof.

In some embodiments, the antigen other than MUC17 is an antigen on the surface of a T cell, a B cell, a natural killer cell, a dendritic cell, a macrophage, a monocyte, or a neutrophil; preferably, the antigen other than MUC17 is selected from: CD96, PD-1, PD-L1, PD-L2, OX40, OX40L, LAG-3, TIM3, VISTA, CD3, CD37, CD36, CD3R, CD3(, CD27, CD28, CD28H, CD16, CD16A, CD32B, VEGF, NKG2D, NKp30, NKp46, NKp44, CD19, CD20, CD40, CD47, 4-1BB, ICOS, OX40, EGFR, EGFRvIII, TNF-alpha, CD33, HER2, HER3, HAS, CD5, CD27, EphA2, EpCAM, MUC1, MUC16, CEA, Claudin18.2, a folate receptor, Claudin6, WT1, NY-ESO-1, MAGE3, ASGPR1, TGFβ-trap, IL-2, IL-15, IL-21, IL-18, or CDH16;
preferably, the multispecific molecule may be bispecific, trispecific, or tetraspecific, and more preferably, the multispecific molecule may be divalent, tetravalent, or hexavalent.

In some embodiments, the multispecific molecule is a tandem scFv, a bifunctional antibody (Db), a single chain bifunctional antibody (scDb), a dual affinity retargeting (DART) antibody, F(ab′)2, a dual variable domain (DVD) antibody, a knobs-into-holes (KiH) antibody, a dock-and-lock (DNL) antibody, a chemically cross-linked antibody, a heteropolymeric nanobody, or a heteroconjugate antibody.

In another aspect, the present application provides a chimeric antigen receptor (CAR), wherein the chimeric antigen receptor at least comprises an extracellular antigen-binding domain, a transmembrane domain, and an intracellular signaling domain; the extracellular antigen-binding domain comprises any one of the foregoing nanobodies or the antigen-binding fragment thereof.

In another aspect, the present application provides an immune effector cell, wherein the immune effector cell expresses the chimeric antigen receptor or comprises a nucleic acid fragment encoding the chimeric antigen receptor; preferably, the immune effector cell is selected from a T cell, a natural killer cell (NK cell), a natural killer T cell (NKT cell), a double negative T cell (DNT cell), a monocyte, a macrophage, a dendritic cell, or a mast cell, and the T cell is preferably selected from a cytotoxic T cell, a regulatory T cell, or a helper T cell; preferably, the immune effector cell is an auto-immune effector cell or an allogeneic immune effector cell.

In another aspect, the present application provides an isolated nucleic acid fragment, wherein the nucleic acid fragment encodes any one of the foregoing nanobodies or the antigen-binding fragment thereof, or the multispecific molecule, or the chimeric antigen receptor.

In a preferred embodiment, the nucleic acid may be a codon-optimized nucleic acid suitable for expression in a host cell. For example, based on the degeneracy of a codon, it still encodes the same protein. Methods for codon optimization depending on the host cell used are well known to those skilled in the art.

In another aspect, the present application provides a vector, wherein the vector comprises the nucleic acid fragment. Preferably, the vector is an expression vector.

Any suitable expression vector may be used. For example, prokaryotic cloning vectors include plasmids from E. coli, such as colEl, pCR1, pBR322, pMB9, pUC, pKSM, and RP4. Prokaryotic vectors also include phage DNA such as M13 and derivatives of other filamentous single-stranded DNA phages. An example of a vector that can be used in yeast is a 2μ plasmid. Suitable vectors for expression in mammalian cells include the following well known derivatives: SV-40, adenoviruses, retrovirus-derived DNA sequences, and shuttle vectors derived from functional mammalian vectors (such as those described above) and combinations of functional plasmids and phage DNAs.

Additional eukaryotic expression vectors are known in the art (e.g., P J. Southern & P. Berg, J. Mol. Appl. Genet, 1:327-341 (1982); Subramani et al., Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), which are incorporated herein by reference in their entirety).

The expression vectors that can be used in the present application comprise at least one expression control sequence operably linked to the DNA sequence or fragment to be expressed. The control sequence is inserted into the vector to control and regulate the expression of the cloned DNA sequence. Examples of useful expression control sequences are the lac system, the trp system, the tac system, the trc system, main operator and promoter regions of a λ phage, the control region of an fd coat protein, a glycolytic promoter of yeast such as a promoter of 3-phosphoglycerate kinase, a promoter of yeast acidic phosphatase such as Pho5, a promoter of yeast α-mating factor, and promoters derived from polyomaviruses, adenoviruses, retroviruses, and simian viruses, such as early and late promoters of SV40 and other sequences known to control gene expression in prokaryotic or eukaryotic cells and viruses thereof or combinations thereof.

In another aspect, the present application provides a host cell, wherein the host cell comprises the vector; preferably, the cell is a prokaryotic cell or a eukaryotic cell, such as a bacterium (E. coli), a fungus (yeast), an insect cell, or a mammalian cell (a CHO cell line or a 293T cell line).

In another aspect, the present application provides a method for preparing any one of the foregoing nanobodies or the antigen-binding fragment thereof, or multispecific molecules, wherein the method comprises culturing the host cell, and isolating a nanobody or an antigen-binding fragment thereof expressed by the cell, or isolating a multispecific molecule expressed by the cell.

In another aspect, the present application provides a method for preparing the immune effector cell, wherein the method comprises introducing a nucleic acid fragment encoding the CAR into the immune effector cell; optionally, the method further comprises initiating expression of the CAR in the immune effector cell.

In another aspect, the present application provides a pharmaceutical composition, wherein the pharmaceutical composition comprises any one of the foregoing nanobodies or the antigen-binding fragment thereof, multispecific antibodies, immune effector cells, nucleic acid fragments, vectors, or host cells, or a product prepared by any one of the foregoing methods; optionally, the pharmaceutical composition further comprises a pharmaceutically acceptable carrier, diluent, or adjuvant.

In some embodiments, the pharmaceutically acceptable carrier is a carrier that does not decrease the viability and the function of immune cells, and does not affect the specific binding of an antibody or an antigen-binding fragment thereof to an antigen, including but not limited to cell culture media, buffers, physiological saline, balanced salt solutions, and the like. Examples of buffers include isotonic phosphates, acetates, citrates, borates, carbonates, and the like. In a specific embodiment, the pharmaceutically acceptable carrier is a phosphate-buffered saline containing 1% serum.

The nanobodies or the antigen-binding fragments thereof and pharmaceutical compositions thereof disclosed herein can be used to treat, ameliorate or prevent a tumor in an individual.

The nanobodies or the antigen-binding fragments thereof and pharmaceutical compositions thereof disclosed herein may be administered in any suitable manner. Preferably, the nanobodies or the antigen-binding fragments thereof and pharmaceutical compositions thereof of the present application are administered by injection (e.g., subcutaneously, intravenously, intratumorally, intraarterially, intramuscularly, intradermally, intraperitoneally, or intrathecally). Preferably, the nanobodies or the antigen-binding fragments thereof and pharmaceutical compositions thereof of the present application are administered intravenously. For the nanobodies or the antigen-binding fragments thereof and pharmaceutical compositions thereof of the present application, suitable pharmaceutically acceptable carriers for injection may include any isotonic carrier, such as physiological saline (water containing about 0.90% w/v NaCl, water containing about 300 mOsm/L NaCl, or about 9.0 g of NaCl per liter of water), NORMOSOL R electrolyte solution (Abbott, Chicago, IL), PLASMA-LYTE A (Baxter, Deerfield, IL), water containing about 5% glucose, or lactated Ringer's solution. In a specific embodiment, the pharmaceutically acceptable carrier is replaced with human serum albumin.

Specifically, the pharmaceutical composition may further comprise a second medicament for treating, ameliorating or preventing a tumor in an individual. For example, various chemotherapeutic agents, such as alkylating agent chemotherapeutic agents, including but not limited to cyclophosphamide, ifosfamide, melphalan, carmustine, lomustine, nimustine, fotemustine, and the like; antimetabolite chemotherapeutic agents, including but not limited to methotrexate, fluorouracil, tegafur, carmofur, doxifluridine, capecitabine, gemcitabine, raltitrexed, and the like; anti-cancer antibiotics, including but not limited to actinomycin D, mitomycin, bleomycin, daunorubicin, adriamycin, epirubicin, pirarubicin, and the like; plant-based chemotherapeutic agents, including but not limited to vincristine, vindesine, vinorelbine, irinotecan, topotecan, paclitaxel, docetaxel, and the like; miscellaneous chemotherapeutic agents, including but not limited to dacarbazine, cisplatin, carboplatin, oxaliplatin, nedaplatin, and the like.

In another aspect, further provided is use of any one of the foregoing nanobodies or antigen-binding fragments, multispecific molecules, immune effector cells, nucleic acid fragments, vectors, host cells, products prepared by any one of the foregoing methods, or pharmaceutical compositions disclosed herein in preparing a medicament for preventing and/or treating a tumor, wherein the tumor may be a tumor that expresses MUC17 on the cell surface, such as a tumor involving the digestive system, for example, gastric cancer, pancreatic cancer, and/or gastroesophageal junction carcinoma.

In another aspect, the present application provides a method for preventing and/or treating a tumor, wherein the method comprises administering to a patient in need thereof an effective amount of any one of the foregoing nanobodies or the antigen-binding fragment thereof, multispecific molecules, immune effector cells, nucleic acid fragments, vectors, host cells, products prepared by any one of the foregoing methods disclosed herein, or pharmaceutical compositions, wherein the tumor may be a tumor that expresses MUC17 on the cell surface, such as a tumor involving the digestive system, for example, gastric cancer, pancreatic cancer, and/or gastroesophageal junction carcinoma.

In another aspect, the present application further provides use of any one of the foregoing nanobodies or the antigen-binding fragment thereof, multispecific molecules, immune effector cells, nucleic acid fragments, vectors, host cells, products prepared by any one of the foregoing methods, or pharmaceutical compositions in preventing and/or treating a tumor, wherein the tumor may be a tumor that expresses MUC17 on the cell surface, such as a tumor involving the digestive system, for example, gastric cancer, pancreatic cancer, and/or gastroesophageal junction carcinoma.

In another aspect, the present application provides a kit, wherein the kit comprises any one of the foregoing nanobodies or the antigen-binding fragment thereof, multispecific antibodies, immune effector cells, nucleic acid fragments, vectors, host cells, products prepared by any one of the foregoing methods, or pharmaceutical compositions.

In another aspect, the present application provides a method for detecting MUC17 expression, comprising contacting a sample to be tested with any one of the foregoing nanobodies or the antigen-binding fragment thereof in a condition allowing formation of a complex by any one of the foregoing nanobodies or the antigen-binding fragment thereof and MUC17.

In another aspect, the present application provides a method for inhibiting the proliferation or migration of a cell expressing MUC17 in vitro, comprising contacting the cell with any one of the foregoing nanobodies or the antigen-binding fragment thereof in a condition allowing formation of a complex by any one of the foregoing nanobodies or the antigen-binding fragment thereof and MUC17.

### Example

The present application will be further described with reference to specific examples, and the advantages and features of the present application will become more apparent with the description. Experimental procedures without specified conditions in the examples are conducted according to conventional conditions or conditions recommended by the manufacturers. Reagents or instruments without specified manufacturers used herein are conventional products that are commercially available.

The examples of the present application are exemplary only, and do not limit the scope of the present application in any way. It will be understood by those skilled in the art that various modifications or substitutions may be made to the technical solutions of the present application in form and details without departing from the spirit and scope of the present application, and that these modifications and substitutions shall fall within the protection scope of the present application.

### Example 1. Preparation of Recombinant Protein and Control Antibody and Purification Method Thereof

### 1.1 Design and Expression of Recombinant Protein

The amino acid sequence (UniProt: Q685J3) containing and encoding the human MUC17 protein extracellular domain truncation was cloned into a His-tagged pTT5 vector (Youbio, VT2202). A plasmid was prepared according to the plasmid extraction kit method and transiently expressed in Expi 293F cells (Gibco, A14527) to give the antigen and the protein for detection used in the examples. The method for preparing the cynomolgus monkey MUC17 extracellular domain truncation protein was the same as the method for preparing the human recombinant protein. The cynomolgus monkey MUC17 sequence was from Uniprot No: AOA2K5WH09. The specific sequence information of the recombinant protein is shown below:

Human MUC17 ECD4131-his (His-tagged human MUC17 protein extracellular domain
fusion protein) (SEQ ID NO: 1):
RTTTCFGDGCQNTASRCKNGGTWDGLKCQCPNLYYGELCEEVVSSIDIGPPETISAQMELTV
TVTSVKFTEELKNHSSQEFQEFKQTFTEQMNIVYSGIPEYVGVNITKLRLGSVVVEHDVLLR
TKYTPEYKTVLDNATEVVKEKITKVTTQQIMINDICSDMMCFNTTGTQVQNITVTQYDPEE
DCRKMAKEYGDYFVVEYRDQKPYCISPCEPGFSVSKNCNLGKCQMSLSGPQCLCVTTETH
WYSGETCNQGTQKSLVYGHHHHHH*
Cyno MUC17 ECD3577-his (His-tagged cynomolgus monkey MUC17 protein
extracellular domain fusion protein) (SEQ ID NO: 2):
PLATIPVSTTSLTSSEGSTISTPSVDISTPVTTSIVGDQCSRCKNGGFWDGLKCQCLTPYYGES
CEEVVNSIDIAPPETVSAQMELTVTVTSVKFTDELKNHSSQEFREFNKTFTEQMNIVYSGIPE
YVGVNITNLRLGSVVVEHDVLLRTKYTPEYKTALDNATEVVKQKITKVTTEQIMTNDNCSA
LMCFNTTGTQVQNITVTQYDPVEECRQKAEEYEDYFLVEYRDQKPYCISPCESGFNASKNC
NHGKCQMSQNGARCLCVTTETHWYSGEDCNQGTQKSHHHHHH*

### 1.2 Recombinant Expression and Purification of MUC17 Control Antibody

The control antibodies used in the examples are all derived from published patent sequences, and the 21D11 and 4C11 antibody sequences are derived from published patent WO2019133961A1. Unless otherwise indicated, the 2D11 and 4C11 control antibodies were for recombinant expression using the human IgG1+κ subtype. The nanobodies and humanized antibodies thereof used in the examples were all for recombinant expression using the human Fc fusion form.

The expression and purification process of the control antibody was as follows: The antibody sequence gene was synthesized and cloned into an expression vector pTT5. Expi293F cells (purchased from Gibco, A14527) were transiently transfected, and the cell supernatant was collected after the cells were cultured on a shaker at 37° C. for 7 days for protein A antibody purification. See “1.3.2 Purification of control antibody by Protein A affinity chromatography” for the purification process. The resulting control antibodies were designated as 2D11-hIgG1 and 4C11-hlgG1. The specific sequence information of the antibodies is shown in Table 1.

TABLE 1
Sequence listing of control antibodies

| Antibody | Sequence | |
|---|---|---|
| name | No. | Sequence |
| 2D11-hIgG1 | SEQ ID | QVQLVESGGGVVQPGRSLRLSCAASGFTFSNHGMHWVRQAPGKC |
| | NO. 3 | LEWVAGIWSDASNKYYAEAVKGRFTISRDTSKNTLYLQMNSLRAE |
| | | DTAVYYCARATYTTGWSYFDYWGQGTLVTVSSASTKGPSVFPLAP |
| | | SSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWL |
| | | NGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK* |
| 2D11-hIgK | SEQ ID | SYELTQSPSVSVSPGQTASITCSGDKLGDKYTSWYQQKPGQSPVLVI |
| | NO. 4 | YHDRKRPSGIPERFSGSNSGNTATLTISGTQAMDEADYYCQAYDRS |
| | | TAWVFGCGTKLTVLRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC* |
| 4C11-hIgG1 | SEQ ID | QVQLVESGGGVVQPGRSLRLSCAASGFTFSSYGMHWVRQAPGKCL |
| | NO. 5 | EWVAVISYDASNKYYASAVKGRFTISRDNSKNTLYLQMNSLRAEDT |
| | | AVYYCARGAYTYGFDYWGQGTLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKLT |
| | | VDKSRWQQGNVFSCSVMHEALHNHYTQKSLSLSPGK* |
| 4C11-hIgK | SEQ ID | EIVLTQSPGTLSLSPGERATLSCRASQSVNRYLAWYQQKPGQAPRLL |
| | NO. 6 | IYGASNRATGIPDRFTGSGSGTDFTLTISRLEPEDFAVYFCHHYGSSIF |
| | | AFGCGTKVEIKRTVAAPSVFIFPPSDEQLKSGTASVVCLLNNFYPRE |
| | | AKVQWKVDNALQSGNSQESVTEQDSKDSTYSLSSTLTLSKADYEK |
| | | HKVYACEVTHQGLSSPVTKSFNRGEC* |
| Human Fc | SEQ ID | EPKSSDKTHTCPPCPAPELLGGPSVFLFPPKPKDTLMISRTPEVTCVV |
| (C220S) | NO. 7 | VDVSHEDPEVKFNWYVDGVEVHNAKTKPREEQYNSTYRVVSVLT |
| sequence | | VLHQDWLNGKEYKCKVSNKALPAPIEKTISKAKGQPREPQVYTLPP |
| | | SREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLS |
| | | PGK* |

### 1.3 Purification of Recombinant Protein and Control Antibody

### 1.3.1 Purification of Recombinant Protein on Nickel Column

After the relevant recombinant protein was constructed and expressed according to the step “1.1 Design and expression of recombinant protein”, purification was performed as follows: The cell expression supernatant sample was centrifuged at high speed to remove impurities, and a nickel column was equilibrated with 20 mM PBS+500 mM NaCl solution and washed with 2-5 column volumes. The culture supernatant was loaded onto a Ni affinity chromatographic column (purchased from GE Healthcare), and meanwhile, the changes in UV absorption value (A280 nm) were monitored using an ultraviolet (UV) detector. The column was washed with an equilibration buffer until the A280 reading dropped to the baseline, then gradient elution was separately performed using imidazole with different concentrations, and each of the elution peaks was collected. The component of the target protein was determined according to the SDS-PAGE gel image. The eluted product was collected and concentrated, which may be further purified by gel chromatography Superdex200 (GE).

The obtained proteins were subjected to electrophoresis and HPLC purity detection, and aliquoted for later use after it was determined to be acceptable. The proteins obtained by purification by this protocol include human MUC17-His and monkey MUC17-His.

### 1.3.2 Purification of Control Antibody by Protein a Affinity Chromatography

The obtained antibody sequences were cloned into eukaryotic expression vectors pTT5 with a human Fc tag, respectively. Expi293F cells were transiently transfected by PEI (Polysciences, 24765-1), cultured for 7 days, and then centrifuged at high speed to collect the cell culture supernatant expressing the antibodies. The Protein A(Bestchrom, AA0273) protein chromatographic column was washed with 3-5 column volumes of 0.1 M NaOH, and then washed with 3-5 column volumes of pure water. The chromatographic column was equilibrated using 3-5 column volumes of 1×PBS (pH 7.4) buffer system as the equilibration buffer. The cell supernatant was loaded on the column at a low flow rate for binding, with the flow rate controlled to allow for a retention time of about 1 min or longer.

After the binding was completed, the chromatographic column was washed with 3-5 column volumes of 1×PBS (pH 7.4) until the UV absorbance fell back to baseline. The sample was eluted with a 50 mM citric acid/sodium citrate (pH 3.0-3.5) buffer, the elution peaks were collected by UV detection, and the eluted product was rapidly adjusted to pH 5-6 with 1 M Tris-HCl (pH 8.0) and temporarily stored. For the eluted product, solution exchange may be performed by methods well known to those skilled in the art, such as ultrafiltration concentration using an ultrafiltration tube to exchange the solution to a desired buffer system, or exchange with a desired buffer system by size exclusion (e.g., G-25 desalination), or removal of polymer components from the eluted product using a high-resolution molecular exclusion column such as Superdex 200 to improve sample purity. After protein A protein affinity purification, an antibody eluted from the chromatographic column with a human Fc tag was collected to give a corresponding control antibody.

### Example 2. Preparation of Nanobody Against MUC17 Protein

### 2.1 Animal Immunization and Serum Titer Assay

2 camels of about 3 years old were selected, and 5 mL of blood was collected before immunization, which was left as the negative control serum. For primary immunization, 0.2 mg of the human MUC17 protein prepared in Example 1 was mixed well with Freund's complete adjuvant (purchased from Sigma, F5881) and then injected subcutaneously in multiple spots into the neck for immunization. After two weeks, a second immunization was performed. 0.1 mg of the human MUC17 protein was mixed with Freund's incomplete adjuvant and then injected subcutaneously in multiple spots into the neck for immunization. After one week, the serum was taken for titer assay. The titer and specificity of the antibody in the serum against human and monkey MUC17 proteins were assayed using enzyme-linked immunosorbent assay (ELISA). The results of the serum titer of the fourth immunization are shown in Table 2 in which the data are OD450 nm values.

TABLE 2
Serum antibody titer after being immunized
with a MUC17 protein assayed by ELISA

OD 450
Batch

| | Animal 1 | Animal 2 | Animal 1 | Animal 2 |
|---|---|---|---|---|
| | Human | Human | Monkey | Monkey |
| Dilution | MUC17 | MUC17 | MUC17 | MUC17 |
| ratio | protein | protein | protein | protein |
| 900 | 1.9 | 2.02 | 1.779 | 1.788 |
| 2700 | 1.974 | 1.845 | 1.914 | 1.701 |
| 8100 | 1.74 | 1.902 | 1.979 | 1.744 |
| 24300 | 1.625 | 1.763 | 1.437 | 1.263 |
| 72900 | 1.355 | 1.363 | 0.948 | 0.777 |
| 218700 | 0.86 | 0.872 | 0.471 | 0.382 |
| 656100 | 0.419 | 0.386 | 0.238 | 0.194 |
| 1968300 | 0.173 | 0.157 | 0.129 | 0.116 |
| 5904900 | 0.097 | 0.074 | 0.104 | 0.093 |
| Blank | 0.038 | 0.037 | 0.084 | 0.08 |

### 2.2 Construction of Nanobody Phage Library

After the immunization was completed, 50 mL of peripheral blood was collected, and PBMCs were isolated using a lymphocyte isolation medium. The total RNA was extracted using Trizol reagent, and cDNA was obtained according to the operations of the reverse transcription kit PrimeScript™ II 1St Strand cDNA Synthesis Kit (Takara, 6210A). The nanobody (VHH) fragments were amplified by nested PCR. After the PCR product was purified, it was ligated into the phage display vector pComb3Xss using a restriction endonuclease SfiI (purchased from NEB, R0123L), and the ligation product was purified. The ligation product was then electrotransformed into TG1 competent cells for 7 times, and 1 mL of SOC medium was immediately added to the cuvette after electroporation to thaw and culture the cells for 1 h, thereby giving 7.3 mL of a TG1 cell thawing product in total. The TG1 bacterium solution was serially diluted by 103 times and 104 times. The number of transformants in the nanobody library was determined. The TG1 bacterium solutions diluted by 103 times and 104 times were coated on a 150 mm plate, and the remaining bacterium solution was coated on 7 150 mm plates. The sizes of the reservoir were calculated to be 1.5×10⁹ and 1.3×10⁹, respectively. 96 samples were sequenced. It was shown that the insertion rate of the library was about 99%, and no repeated sequence existed, thereby indicating that the construction of the phage library was successful.

### 2.3 Panning of Nanobodies Against MUC17

This patent uses a cross-panning method using the human and monkey MUC17 proteins. For the first round of biopanning, three tubes of A, B, and C were prepared, 100 μL of streptavidin-conjugated Dynabeads (purchased from Invitrogen) and the phage antibody library described above were first added to tube A, and 100 μL of streptavidin-conjugated Dynabeads were first added to tube B. Then a blocking buffer, i.e., PBS phosphate-buffered saline containing 20% (w/v) skim milk powder, was separately added to the three tubes for blocking at room temperature for 2 h. The liquid in tube C was discarded, the supernatant collected after centrifugation of tube A was added, then 4 g of biotinylated human MUC17-His protein was added, and biotinylation was performed according to kit instructions (purchased from Dojindo, LK03). The mixture was incubated at room temperature for 2 h while shaking. Moreover, a control tube was set, only non-biotinylated human MUC17-His protein was added, and the mixture was incubated at room temperature for 1 h while shaking. Tube B was centrifuged to give the blocked magnetic beads, the incubated mixed solution was added, and the mixture was incubated at room temperature for 15 min while shaking. The tube was placed on the magnetic rack for 30 s, washed 10 times with 1 mL of PBST, i.e., blocking buffer containing 0.01% (v/v) Tween-20, and washed 1 time with PBS buffer. After washing, 500 μL of 10 g/mL pancreatin was added to each tube, and the mixture was incubated at 37° C. for 15 min to elute phages binding to the biotinylated human MUC17-His protein. 250 μL of a pancreatin solution was added to 4 mL of E. coli TG1 (purchased from LUCIGEN) in the logarithmic growth phase, and the mixture was incubated at 37° C. for 30 min to give a TG1 culture solution. The TG1 culture solution was serially diluted, coated on a plate, and cultured at 37° C. overnight. The number of the resulting clones binding to the biotinylated human MUC17-His protein and clones of the control tube was calculated, and 48 clones were selected for sequencing. Meanwhile, the clones on the plate were washed with 2YT medium (purchased from Sangon, 2YT medium was prepared by adding 31 g of 2YT medium powder to 1 μL of water, adjusting the pH to 7.0 with NaOH, and autoclaving), collected, inoculated into a fresh medium, and cultured at 37° C. to the logarithmic phase. Helper phages M13K07 (purchased from NEB, Cat. No. N0315S) were added with a ratio of helper phage to E. coli TG1 of 20:1. The mixture was mixed well and left to stand at 37° C. for 30 min. Then the mixture was cultured at 37° C. for 30 min while shaking and centrifuged at 4000 rpm for 10 min. The cells were collected, a fresh 2YT medium containing ampicillin and kanamycin resistance was added, and the mixture was cultured at 30° C. overnight while shaking. The mixture was centrifuged at 5000 rpm for 20 min, the supernatant was collected, ¼ of the supernatant volume of a 2.5 M NaCl solution containing 20% PEG6000 was added, and the mixture was left on ice overnight. The mixture was centrifuged at 5000 rpm at 4° C. for 30 min, and the phage pellet was collected and dissolved in PBS buffer. The mixture was centrifuged at 10000 rpm for 10 min to remove the residual cell debris, and the supernatant was collected for the next round of biopanning.

The steps of the second round of panning were consistent with those of the first round. In the second round, VHH antibodies that specifically bind to the biotinylated monkey MUC17-His protein were enriched. After multiple rounds of panning, the positive phages were continuously enriched in the panning process, so that a nanobody with good specificity and high affinity was screened out.

### 2.4 Screening of Positive Phage Clones by Enzyme-Linked Immunosorbent Assay

From the second and three rounds of plates, single clones were selected and cultured in a 96-well plate. 200 μL of 2YT medium containing antibiotics and 1% glucose was added to each well, and the mixture was cultured at 250 rpm at 37° C. overnight while shaking. 10 μL of the overnight culture was added to 100 μL of 2YT medium containing antibiotics and 0.5% glucose, and the mixture was cultured until the OD600 was 0.4-0.6. Helper phages were added at an infection ratio of 20:1, and the mixture was left to stand at 37° C. for 30 min. Then the mixture was cultured at 37° C. for 30 min while shaking, 400 μL of 2YT medium containing antibiotics was added, and the mixture was cultured at 30° C. overnight. The next day, the mixture was centrifuged at 5000 rpm at 4° C. for 20 min, and the resulting supernatant was used for monoclonal ELISA identification.

The human and monkey MUC17 proteins were diluted with carbonate buffer at pH 9.6 to a final concentration of 2 μg/mL, added to enzyme-labeled wells at 50 μL/well, and coated at 4° C. overnight. Then 50 μL of the phage culture bacterium solution supernatant and a horseradish peroxidase-labeled anti-M13 antibody (purchased from Sino Biological, 11973-MM05T-H) diluted at a ratio of 1:4000 were added to each well. A TMB color-developing solution (purchased from KPL, 52-00-02) was added for color developing after washing the plates, and the optical density was measured at 450 nm. Positive clones binding to both human and monkey MUC17 proteins were selected for FACS detection.

### 2.5 Screening of Phage Clone Binding to Cell by Flow Cytometry Assay (FACS)

A nucleotide sequence encoding the monkey MUC17 fragment was cloned into a pcDNA5 vector (purchased from General). A plasmid was prepared and an overexpression cell line was constructed. A monoclonal cell line with a relatively high fluorescence intensity was selected for subsequent detection. The constructed overexpression cell line was designated as FlpinCHO-Cyno(3597-3964)-D2. The endogenous tumor cell line NUGC4 (Nanjing Cobioer, Cat. No. CBP60493) and the FlpinCHO-Cyno(3597-3964)-D2 overexpression cell strain were expanded in a T-175 cell culture flask until the confluence was 90%. The medium was completely pipetted off. The cells were washed 1 time with PBS buffer, then treated with Trypsin-EDTA (purchased from Gibco, Cat. No. 25200072) and collected. After counting, the cells were washed 2 times with PBS phosphate-buffered saline, diluted to 2×10⁶ cells per mL, and added to a 96-well FACS reaction plate at 50 μL/well. 1% (w/w) fetal bovine serum was added to the PBS phosphate-buffered saline as an FACS buffer, and the plate was centrifuged at 1000 rpm at 4° C. and washed 2 times. 50 i L of the phage supernatant was added to each well, and the mixture was incubated on ice for 1 h. After the plate was centrifuged and washed 3 times with the FACS buffer, 50 μL of an anti-M13 antibody (Sino Biological, Cat. No. 11973-MM05T) diluted at a ratio of 1:1000 was added to each well, and the mixture was incubated on ice for 1 h. After the plate was centrifuged and washed 3 times with the FACS buffer, a fluorescently (Alexa 647)-labeled secondary antibody (Jackson Immuno, Cat. No. 115-605-003) was added to each well, and the mixture was incubated on ice for 1 h. The plate was centrifuged and washed 3 times with the FACS buffer. The cells were suspended in 100 μL of the FACS buffer and tested on an FACS machine, and the results were analyzed.

Through multiple rounds of optimization and screening, 5 positive clones capable of recognizing human and monkey MUC17 simultaneously were obtained and designated as Lab315, Lab316, Lab320, Lab33E, and Lab332, respectively. The CDRs of their sequences were analyzed using KABAT, Chothia, or IM4GT software, respectively. The corresponding sequence information is shown in Tables 3-4 below, wherein Table 3 shows the amino acid sequences of 5 nanobody molecules, and Table 4 shows the results of IM4GT, Kabat, and Chothia analysis of the CDRs of 5 nanobody molecules.

TABLE 3
Amino acid specific sequence information of anti-MUC17 nanobody

| Antibody | | |
|---|---|---|
| name | Sequence No. | Amino acid sequence |
| Lab315 | SEQ ID NO. 8 | DVQLQASGGGAVQAGGSLRLSCAASGDITRRCMGWFRQAPGK |
| | | EREGVAGISEDGITIYADSVKGRFTISKDNAKNSLYLQMNSLAPE |
| | | DTANYSCAASILNRVGCRQLSYEYIYRGQGTQVTVSS |
| Lab316 | SEQ ID NO. 9 | DVQLQASGGGSVQAGGSLRLSCAASDNTFSRGSMSWFRQAPGK |
| | | EREGVAIISSDGWTGYADSVKGRFTISQDNAKNTLYLQMIGLKPD |
| | | DTAMYYCAASFLVLRALNPKAWHYWGQGTQVTVSS |
| Lab320 | SEQ ID NO. 10 | DVQLQASGGGSVQSGGSLRLSCAASGFPSSRGTMAWFRQAPGR |
| | | EREGVAFINSDGRTSYAESVKGRFTISRDNALSTLWLQMNSLKPE |
| | | DTGMYYCAASLLYLMTVNAAAYPYWGQGTQVTVSS |
| Lab331 | SEQ ID NO. 11 | DVQLQASGGGSVQAGGSLRLSCAASGYTSNRYSMAWFRQAPG |
| | | KEREVVAFIWTDGGRTYYADSVKGRLTISHDNAKNTLYLQMDSL |
| | | KPEDTAMYYCAASSLLLQTINPRAWPYWGQGTQVTVSS |
| Lab332 | SEQ ID NO. 12 | EVQLQASGGGSVQAGGSLRVSCAASGDTFSRGSWGWFRQRPG |
| | | KEREAVALISSDGWTFYADSVKGRFTISQDNTKNTLYLQMISLRP |
| | | EDTAMYYCAASVLVLRTLNPKAWHHWGQGTQVTVSS |

TABLE 4
Analysis of CDR specific sequence information of MUC17 nanobody by IMGT,
KABAT, and Chothia software
IMGT Analysis

| Antibody | Sequence | | | | Sequence | |
|---|---|---|---|---|---|---|
| No. | No. | CDR1 | Sequence No. | CDR2 | No. | CDR3 |
| Lab315 | SEQ ID | GDITRRC | SEQ ID NO. 14 | ISEDGIT | SEQ ID | AASILNRVGCRQ |
| | NO. 13 | | | | NO. 15 | LSYEYIY |
| Lab316 | SEQ ID | DNTFSRGS | SEQ ID NO. 17 | ISSDGWT | SEQ ID | AASFLVLRALNP |
| | NO. 16 | | | | NO. 18 | KAWHY |
| Lab320 | SEQ ID | GFPSSRGT | SEQ ID NO. 20 | INSDGRT | SEQ ID | AASLLYLMTVNA |
| | NO. 19 | | | | NO. 21 | AAYPY |
| Lab331 | SEQ ID | GYTSNRYS | SEQ ID NO. 23 | IWTDGGRT | SEQ ID | AASSLLLQTINPR |
| | NO. 22 | | | | NO. 24 | AWPY |
| Lab332 | SEQ ID | GDTFSRGS | SEQ ID NO. 26 | ISSDGWT | SEQ ID | AASVLVLRTLNP |
| | NO. 25 | | | | NO. 27 | KAWHH |

Kabat analysis

| Antibody | Sequence | | | | Sequence | |
|---|---|---|---|---|---|---|
| No. | No. | CDR1 | Sequence No. | CDR2 | No. | CDR3 |
| Lab315 | SEQ ID | RCMG | SEQ ID NO. 29 | GISEDGITIY | SEQ ID | SILNRVGCRQLS |
| | NO. 28 | | | ADSVKG | NO. 30 | YEYIY |
| Lab316 | SEQ ID | RGSMS | SEQ ID NO. 32 | IISSDGWTG | SEQ ID | SFLVLRALNPKA |
| | NO. 31 | | | YADSVKG | NO. 33 | WHY |
| Lab320 | SEQ ID | RGTMA | SEQ ID NO. 35 | FINSDGRTS | SEQ ID | SLLYLMTVNAAA |
| | NO. 34 | | | YAESVKG | NO. 36 | YPY |
| Lab331 | SEQ ID | RYSMA | SEQ ID NO. 38 | FIWTDGGRT | SEQ ID | SSLLLQTINPRAW |
| | NO. 37 | | | YYADSVKG | NO. 39 | PY |
| Lab332 | SEQ ID | RGSWG | SEQ ID NO. 41 | LISSDGWTF | SEQ ID | SVLVLRTLNPKA |
| | NO. 40 | | | YADSVKG | NO. 42 | WHH |

Chothia analysis

| Antibody | Sequence | | | | Sequence | |
|---|---|---|---|---|---|---|
| No. | No. | CDR1 | Sequence No. | CDR2 | No. | CDR3 |
| Lab315 | SEQ ID | GDITRR | SEQ ID NO. 44 | SEDGI | SEQ ID | SILNRVGCRQLS |
| | NO. 43 | | | | NO. 45 | YEYIY |
| Lab316 | SEQ ID | DNTFSRG | SEQ ID NO. 47 | SSDGW | SEQ ID | SFLVLRALNPKA |
| | NO. 46 | | | | NO. 48 | WHY |
| Lab320 | SEQ ID | GFPSSRG | SEQ ID NO. 50 | NSDGR | SEQ ID | SLLYLMTVNAAA |
| | NO. 49 | | | | NO. 51 | YPY |
| Lab331 | SEQ ID | GYTSNRY | SEQ ID NO. 53 | WTDGGR | SEQ ID | SSLLLQTINPRAW |
| | NO. 52 | | | | NO. 54 | PY |
| Lab332 | SEQ ID | GDTFSRG | SEQ ID NO. 56 | SSDGW | SEQ ID | SVLVLRTLNPKA |
| | NO. 55 | | | | NO. 57 | WHH |

### Example 3. Expression and Purification of Recombinant Nanobody and Assay on Affinity

### 3.1 Expression and Purification of Recombinant Nanobody

The obtained nanobody sequences were cloned into eukaryotic expression vectors pTT5 with an Fe tag, respectively. Then Expi293F cells (purchased from Gibco, A14527) were transiently transfected by PEI, cultured for 7 days, and then centrifuged at high speed to collect the cell culture supernatant expressing the antibodies. The antibodies were purified according to the purification method described in Example 1.3.2 to give corresponding recombinant nanobodies which were designated as Lab315-huFc, Lab316-huFc, Lab320-huFc, Lab331-huFc, and Lab332-huFc, respectively. The purified antibody was subjected to purity detection using an SEC-HPLC method, and aliquoted for later use after it was determined to be acceptable.

### 3.2 Assay on Binding of Recombinant Antibodies to Human MUC17-his Protein by Enzyme-Linked Immunosorbent Assay (ELISA)

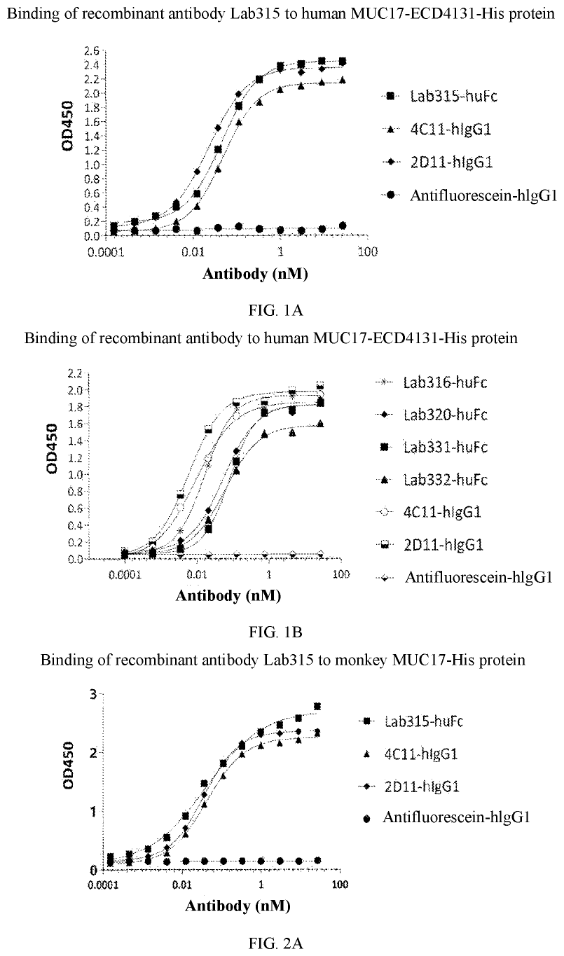

A human MUC17-his protein was diluted with PBS to a final concentration of 2 g/mL and then added to a 96-well ELISA plate at 50 L/well. The plate was incubated at 4° C. overnight. The next day, the plate was washed 2 times with PBST, and a blocking buffer [PBS+2% (w/w) BSA] was added for blocking at room temperature for 2 h. The blocking buffer was discarded, and the recombinant antibody and positive and negative control antibody with a starting concentration of 100 nM serially diluted by 3 folds were added at 50 μL/well. After incubation at 37° C. for 1 h, the plate was washed 3 times with PBST. A horseradish peroxidase (HRP)-labeled secondary antibody (purchased from Merck, Cat. No. AP113P) was added for incubation at 37° C. for 1 h, and the plate was washed 5 times with PBST. A TMB substrate was added at 50 μL/well for incubation at room temperature for 10 min, and then a stop solution (1.0 M HCl) was added at 50 L/well. The OD₄₅₀ nm values were read by an ELISA plate reader (Multimode Plate Reader, EnSight, purchased from Perkin Elmer). The binding activities of the recombinant antibodies Lab315-huFc, Lab316-huFc, Lab320-huFc, Lab331-huFc, and Lab332-huFc with human MUC17 protein are shown in FIGS. 1A-1B, indicating that all the purified antibodies bind efficiently to human MUC17-his protein.

### 3.3 Assay on Binding of Recombinant Antibodies to Monkey MUC17-his by Enzyme-Linked Immunosorbent Assay (ELISA)

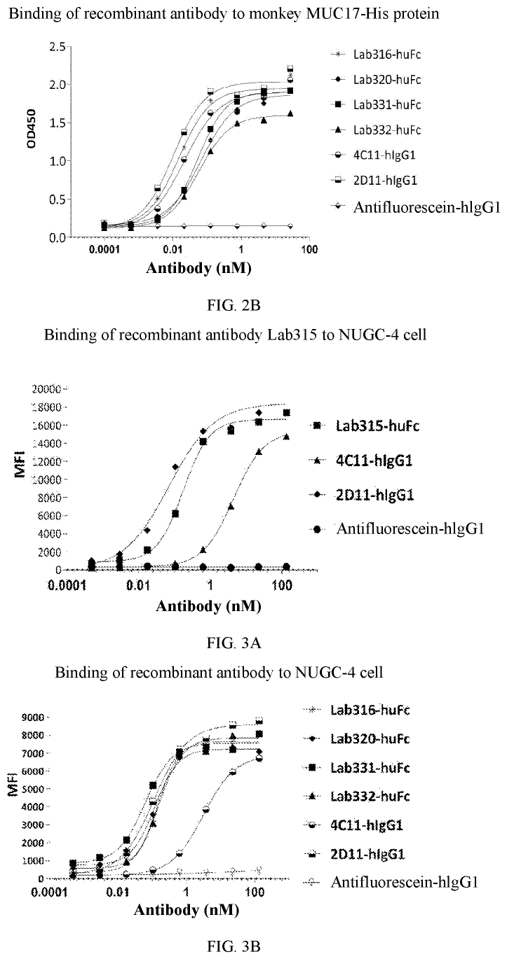

The monkey MUC17-his protein was subjected to ELISA and data analysis according to the method described in Example 3.2. The analysis results are shown in FIGS. 2A-2B. The recombinant antibodies Lab315-huFc, Lab316-huFc, Lab320-huFc, Lab331-huFc, and Lab332-huFc have a relatively good binding activity with the monkey MUC17-his protein.

### 3.4 Assay on Binding of Recombinant Antibodies to Human MUC17 by Flow Cytometry Assay (FACS)

The endogenous tumor cells NUGC4 were expanded to the logarithmic growth phase in a T-175 culture flask, the medium was removed by pipetting, and the cells were washed 2 times with a PBS buffer and digested with trypsin. Then a complete medium was added to stop the digestion, and the cells were blown into a single cell suspension. After counting, the cells were centrifuged, and the cell pellet was resuspended to 2×10⁶ cells/mL in an FACS buffer (PBS+2% fetal bovine serum), and added to a 96-well FACS reaction plate at 100 L/well. The plate was centrifuged, and the supernatant was discarded. An antibody sample to be tested (diluted in a 3-fold gradient from a starting concentration of 100 nM) was added at 50 L/well and uniformly mixed with the cells, and the mixture was incubated at 4° C. for 1 h. After the plate was centrifuged and washed 3 times with a PBS buffer, 50 μL of Alexa Fluor® 647 AffiniPure Goat Anti-Human IgG, Fc7 fragment specific-labeled secondary antibodies (purchased from Jackson, Cat. No. 109-605-098) were added to each well, and the mixture was incubated at 4° C. for 1 h. The results were tested and analyzed by FACS (FACS Canto™, purchased from BD) after the plate was centrifuged and washed 3 times with a PBS buffer and resuspended with 100 μL of PBS. Data analysis was performed by software (FlowJo) to obtain the mean fluorescence intensity (MFI) of the cells. Then, analysis was performed by software (GraphPad Prism8), data were fitted, and EC50 values were calculated. As shown in FIGS. 3A-3B, the recombinant antibodies Lab315-huFc, Lab316-huFc, Lab320-huFc, Lab331-huFc, and Lab332-huFc are all capable of binding specifically to NUGC-4 cells.

### 3.5 Assay on Binding of Recombinant Antibodies to Monkey MUC17 by Flow Cytometry Assay (FACS)

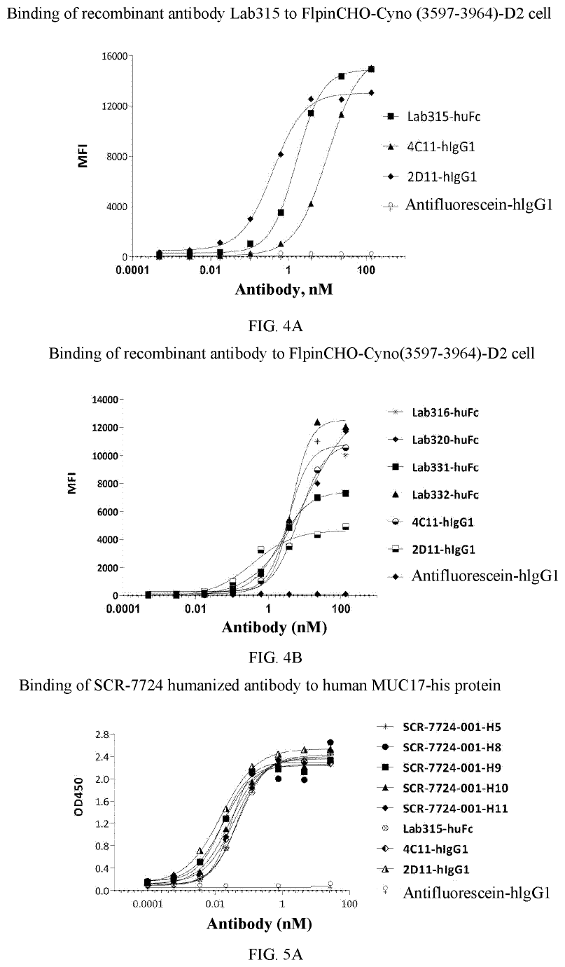

FlpinCHO-Cyno(3597-3964)-D2 recombinant cells were subjected to the FACS assay and data analysis according to the method described in Example 3.4. The results are shown in FIGS. 4A-4B. The recombinant antibodies Lab315-huFc, Lab316-huFc, Lab320-huFc, Lab331-huFc, and Lab332-huFc are capable of binding effectively to FlpinCHO-Cyno(3597-3964)-D2 cells.

### 3.6 Assay on Affinity of Recombinant Antibodies

The strength of antibody-antigen binding was assayed with a BIAcore 8K instrument using an Protein A capture method. First, Protein A was immobilized onto a CM4 chip (purchased from GE, BR-1005-34) using an amino coupling method. According to the instruction of the Amine Coupling Kit (purchased from GE, BR100633), NHS and EDC were mixed with HBS-EP+ pH 7.4 as a mobile phase to activate the chip for about 600 s; the Protein A was diluted to 50 g/mL with 10 mM sodium acetate (pH 4.5) and injected for 600 s, and finally, the remaining activated sites were blocked with ethanolamine. Then, the affinity of the antibody for the antigen was assayed using a multi-cycle kinetic method; in each cycle, first, the antibody to be tested was captured using the Protein A chip, and then a single concentration of antigen protein was injected. The association and dissociation processes of the antibody with the antigen protein were recorded, and finally, the chip was regenerated using Glycine pH 1.5, wherein the mobile phase was HBS-EP+ (10 mM HEPES, 150 mM NaCl, 3 mM EDTA, 0.05% surfactant P20), the flow rate was 30 L/min, the regeneration time was 30 s, and the assay temperature was 25° C. Finally, according to a 1:1 binding model, the data were analyzed, and the antibody-antigen binding kinetics parameters, including the association rate constant Ka, the dissociation rate constant Kd, the equilibrium dissociation constant KD, and the maximum binding signal Rmax, were fitted. The association rate (Ka), dissociation rate (Kd), and binding affinity (KD) of the recombinant antibodies Lab315-huFc, Lab316-huFc, Lab320-huFc, Lab331-huFc, and Lab332-huFc with the human MUC17 protein are shown in Table 5.

TABLE 5
Assay on affinity of recombinant antibodies
for human MUC17 protein by SPR (Biacore)

| | Human MUC17-his protein | | |
|---|---|---|---|
| Antibody name | ka (1/Ms) | kd (1/s) | KD (M) |
| Lab315-huFc | 1.69E+05 | 6.47E−05 | 3.83E−10 |
| Lab316-huFc | 2.65E+05 | 3.43E−05 | 1.29E−10 |
| Lab320-huFc | 1.90E+05 | 1.06E−04 | 5.56E−10 |
| Lab331-huFc | 8.00E+05 | 7.67E−05 | 9.58E−11 |
| Lab332-huFc | 4.91E+05 | 1.18E−04 | 2.39E−10 |
| 2D11-hIgG1 | 1.06E+05 | 7.53E−04 | 7.12E−09 |

### Example 4. Humanization of Nanobody

### 4.1 Humanization Design for Nanobody

By alignment with the IMGT database (http://imgt.cines.fr) for human antibody variable region germline gene sequences, germline genes, with high homology to the nanobodies, from heavy chain variable regions were selected as templates, and CDR sequences of the nanobodies based on the IMGT or KABAT naming method were separately grafted into corresponding humanized templates to form variable region sequences in the order of “FR1-CDR1-FR2-CDR2-FR3-CDR3-FR4”.

The CDR sequences of the Lab315 antibody were defined based on the IMGT scheme, and the humanized templates thereof were IGHV3-9*01 and IGHJ3*01. The Lab315 antibody was redesignated as SCR-7724 during the humanization, and designated as SCR-7724-001-H after CDR grafting.

The CDR sequences of the Lab316 antibody were defined based on the Kabat scheme, and the humanized templates thereof were IGHV3-7*01 and IGHJ3*01. The Lab316 antibody was redesignated as SCR-7782 during the humanization, and designated as SCR-7782-001-H after CDR grafting.

The CDR sequences of the Lab320 antibody were defined based on the Kabat scheme, and the humanized templates thereof were IGHV3-7*01 and IGHJ3*01. The Lab320 antibody was redesignated as SCR-7783 during the humanization, and designated as SCR-7783-001-H after CDR grafting.

The CDR sequences of the Lab331 antibody were defined based on the IMGT scheme, and the humanized templates thereof were IGHV3-30*02 and IGHJ3*01. The Lab331 antibody was redesignated as SCR-7784 during the humanization, and designated as SCR-7784-001-H after CDR grafting.

The CDR sequences of the Lab332 antibody were defined based on the Kabat scheme, and the humanized templates thereof were IGHV3-30*02 and IGHJ3*01. The Lab332 antibody was redesignated as SCR-7785 during the humanization, and designated as SCR-7785-001-H after CDR grafting.

CDRs of the antibodies SCR-7724, SCR-7782, SCR-7783, SCR-7784, and SCR-7785 were respectively grafted into the humanized templates thereof to give humanized antibodies. The amino acid sequences of the humanized templates thereof and the humanized antibody sequences after CDR grafting are shown in Table 6.

TABLE 6
Amino acid specific sequence information of humanized template and humanized
antibody

| | Sequence | |
|---|---|---|
| Name | No. | Amino acid sequence |
| IGHV3-9*01 | SEQ ID | EVQLVESGGGLVQPGRSLRLSCAASGFTFDDYAMH |
| template | NO. 58 | WVRQAPGKGLEWVSGISWNSGSIGYADSVKGRFTI |
| | | SRDNAKNSLYLQMNSLRAEDTALYYCAKD |
| IGHV3-7*01 | SEQ ID | EVQLVESGGGLVQPGGSLRLSCAASGFTFSSYWMS |
| template | NO. 59 | WVRQAPGKGLEWVANIKQDGSEKYYVDSVKGRFT |
| | | ISRDNAKNSLYLQMNSLRAEDTAVYYCAR |
| IGHV3-30*02 | SEQ ID | QVQLVESGGGVVQPGGSLRLSCAASGFTFSSYGMH |
| template | NO. 60 | WVRQAPGKGLEWVAFIRYDGSNKYYADSVKGRFTI |
| | | SRDNSKNTLYLQMNSLRAEDTAVYYCAK |
| FR4-IGHJ3*01 | SEQ ID | WGQGTMVTVSS |
| template | NO. 61 | |
| SCR-7724-001-H | SEQ ID | EVQLVESGGGLVQPGRSLRLSCAASGDITRRCMHW |
| (IGHV3-9*01 Graft) | NO. 62 | VRQAPGKGLEWVSGISEDGITGYADSVKGRFTISRD |
| | | NAKNSLYLQMNSLRAEDTALYYCAASILNRVGCRQ |
| | | LSYEYIYWGQGTMVTVSS |
| SCR-7782-001-H | SEQ ID | EVQLVESGGGLVQPGGSLRLSCAASGFTFSRGSMS |
| (IGHV3-7*01 Graft) | NO. 63 | WVRQAPGKGLEWVAIISSDGWTGYADSVKGRFTIS |
| | | RDNAKNSLYLQMNSLRAEDTAVYYCARSFLVLRAL |
| | | NPKAWHYWGQGTMVTVSS |
| SCR-7783-001-H | SEQ ID | EVQLVESGGGLVQPGGSLRLSCAASGFTFSRGTMA |
| (IGHV3-7*01 Graft) | NO. 64 | WVRQAPGKGLEWVAFINSDGRTSYAESVKGRFTIS |
| | | RDNAKNSLYLQMNSLRAEDTAVYYCARSLLYLMT |
| | | VNAAAYPYWGQGTMVTVSS |
| SCR-7784-001-H | SEQ ID | QVQLVESGGGVVQPGGSLRLSCAASGYTSNRYSM |
| (IGHV3-30*02 | NO. 65 | HWVRQAPGKGLEWVAFIWTDGGRTYYADSVKGRF |
| Graft) | | TISRDNSKNTLYLQMNSLRAEDTAVYYCAASSLLLQ |
| | | TINPRAWPYWGQGTMVTVSS |
| SCR-7785-001-H | SEQ ID | QVQLVESGGGVVQPGGSLRLSCAASGFTFSRGSWG |
| (IGHV3-30*02 | NO. 66 | WVRQAPGKGLEWVALISSDGWTFYADSVKGRFTIS |
| Graft) | | RDNSKNTLYLQMNSLRAEDTAVYYCAKSVLVLRTL |
| | | NPKAWHHWGQGTMVTVSS |

### 4.2. Back Mutation Design for SCR-7724 Humanized Antibodies

Key amino acids in the FR sequence of the SCR-7724 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 7-8.

TABLE 7
Mutation point of back mutation of SCR-7724 humanized antibody

| Antibody name | Mutation point |
|---|---|
| SCR-7724-001-H5 | H34G, V36F, G43E, L44R, W46G, S48A, G57I, |
| Graft(IGHV3-9*01) | R70K, Y93S, W114R |
| SCR-7724-001-H8 | H34G, V36F, G43E, L44R, W46G, S48A, W114R |
| Graft(IGHV3-9*01) | |
| SCR-7724-001-H9 | H34G, V36F, G43E, L44R, W46G, S48A, Y93S, |
| Graft(IGHV3-9*01) | W114R |
| SCR-7724-001-H10 | H34G, V36F, G43E, L44R, W46G, S48A, R70K, |
| Graft(IGHV3-9*01) | Y93S, W114R |
| SCR-7724-001-H11 | H34G, V36F, G43E, L44R, W46G, S48A, R70K, |
| Graft(IGHV3-9*01) | Y93S, W114R, M119Q |

Note:
Graft(IGHV3-9*01) denotes that the CDRs of the nanobody are grafted into the human germline FR sequences; and H34G denotes that H at position 34 of Graft(IGHV3-9*01) is mutated to G, and so on for others.

TABLE 8
Amino acid sequences of SCR-7724 humanized antibodies after back mutation

| Antibody name | Sequence No. | Amino acid sequence |
|---|---|---|
| SCR-7724-001-H5 | SEQ ID NO. 67 | EVQLVESGGGLVQPGRSLRLSCAASGDITRRCMGWFRQAP |
| | | GKEREGVAGISEDGITIYADSVKGRFTISKDNAKNSLYLQM |
| | | NSLRAEDTALYSCAASILNRVGCRQLSYEYIYRGQGTMVT |
| | | VSS |
| SCR-7724-001-H8 | SEQ ID NO. 68 | EVQLVESGGGLVQPGRSLRLSCAASGDITRRCMGWFRQAP |
| | | GKEREGVAGISEDGITGYADSVKGRFTISRDNAKNSLYLQ |
| | | MNSLRAEDTALYYCAASILNRVGCRQLSYEYIYRGQGTM |
| | | VTVSS |
| SCR-7724-001-H9 | SEQ ID NO. 69 | EVQLVESGGGLVQPGRSLRLSCAASGDITRRCMGWFRQAP |
| | | GKEREGVAGISEDGITGYADSVKGRFTISRDNAKNSLYLQ |
| | | MNSLRAEDTALYSCAASILNRVGCRQLSYEYIYRGQGTMV |
| | | TVSS |
| SCR-7724-001-H10 | SEQ ID NO. 70 | EVQLVESGGGLVQPGRSLRLSCAASGDITRRCMGWFRQAP |
| | | GKEREGVAGISEDGITGYADSVKGRFTISKDNAKNSLYLQ |
| | | MNSLRAEDTALYSCAASILNRVGCRQLSYEYIYRGQGTMV |
| | | TVSS |
| SCR-7724-001-H11 | SEQ ID NO. 71 | EVQLVESGGGLVQPGRSLRLSCAASGDITRRCMGWFRQAP |
| | | GKEREGVAGISEDGITGYADSVKGRFTISKDNAKNSLYLQ |
| | | MNSLRAEDTALYSCAASILNRVGCRQLSYEYIYRGQGTQV |
| | | TVSS |

### 4.3. Back Mutation Design for SCR-7782 Humanized Antibodies

Key amino acids in the FR sequence of the SCR-7782 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 9-10.

TABLE 9
Mutation point of back mutation of SCR-7782 humanized antibody

| Antibody name | Mutation point |
|---|---|
| SCR-7782-001-H1 | V37F, G44E, L45R, W47G, R97A |
| Graft(IGHV3-7*01) | |
| SCR-7782-001-H2 | V37F, G44E, L45R, W47G, R71Q, R97A |
| Graft(IGHV3-7*01) | |
| SCR-7782-001-H3 | F27N, V37F, G44E, L45R, W47G, R71Q, R97A |
| Graft(IGHV3-7*01) | |
| SCR-7782-001-H4 | E1D, G26D, F27N, V37F, G44E, L45R, W47G, |
| Graft(IGHV3-7*01) | R71Q, R97A |
| SCR-7782-001-H5 | E1D, V5Q, E6A, G26D, F27N, V37F, G44E, |
| Graft(IGHV3-7*01) | L45R, W47G, R71Q, R97A |
| SCR-7782-001-H6 | E1D, G26D, F27N, V37F, G44E, L45R, W47G, |
| Graft(IGHV3-7*01) | R71Q, V92M, R97A, M118Q |

Note:
Graft(IGHV3-7*01) denotes that the CDRs of the nanobody are grafted into the human germline FR sequences; V37F denotes that V at position 37 of Graft(IGHV3-7*01) is mutated to F, and so on for others.

TABLE 10
Amino acid sequences of SCR-7782 humanized antibodies after back mutation

| | Sequence | |
|---|---|---|
| Antibody name | No. | Amino acid sequence |
| SCR-7782-001-H1 | SEQ ID | EVQLVESGGGLVQPGGSLRLSCAASGFTFSRGSMSWFRQAPG |
| | NO. 72 | KEREGVAIISSDGWTGYADSVKGRFTISRDNAKNSLYLQMNSL |
| | | RAEDTAVYYCAASFLVLRALNPKAWHYWGQGTMVTVSS |
| SCR-7782-001-H2 | SEQ ID | EVQLVESGGGLVQPGGSLRLSCAASGFTFSRGSMSWFRQAPG |
| | NO. 73 | KEREGVAIISSDGWTGYADSVKGRFTISQDNAKNSLYLQMNSL |
| | | RAEDTAVYYCAASFLVLRALNPKAWHYWGQGTMVTVSS |
| SCR-7782-001-H3 | SEQ ID | EVQLVESGGGLVQPGGSLRLSCAASGNTFSRGSMSWFRQAPG |
| | NO. 74 | KEREGVAIISSDGWTGYADSVKGRFTISQDNAKNSLYLQMNSL |
| | | RAEDTAVYYCAASFLVLRALNPKAWHYWGQGTMVTVSS |
| SCR-7782-001-H4 | SEQ ID | DVQLVESGGGLVQPGGSLRLSCAASDNTFSRGSMSWFRQAPG |
| | NO. 75 | KEREGVAIISSDGWTGYADSVKGRFTISQDNAKNSLYLQMNSL |
| | | RAEDTAVYYCAASFLVLRALNPKAWHYWGQGTMVTVSS |
| SCR-7782-001-H5 | SEQ ID | DVQLQASGGGLVQPGGSLRLSCAASDNTFSRGSMSWFRQAP |
| | NO. 76 | GKEREGVAIISSDGWTGYADSVKGRFTISQDNAKNSLYLQMN |
| | | SLRAEDTAVYYCAASFLVLRALNPKAWHYWGQGTMVTVSS |
| SCR-7782-001-H6 | SEQ ID | DVQLVESGGGLVQPGGSLRLSCAASDNTFSRGSMSWFRQAPG |
| | NO. 77 | KEREGVAIISSDGWTGYADSVKGRFTISQDNAKNSLYLQMNSL |
| | | RAEDTAMYYCAASFLVLRALNPKAWHYWGQGTQVTVSS |

### 4.4. Back Mutation Design for SCR-7783 Humanized Antibodies

Key amino acids in the FR sequence of the SCR-7783 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 11-12.

TABLE 11
Mutation point of back mutation of SCR-7783 humanized antibody

| Antibody name | Mutation point |
|---|---|
| SCR-7783-001-H1 | V37F, G44E, L45R, W47G, R97A |
| Graft(IGHV3-7*01) | |
| SCR-7783-001-H2 | T28P, F29S, V37F, G44E, L45R, W47G, R97A |
| Graft(IGHV3-7*01) | |
| SCR-7783-001-H3 | E1D, T28P, F29S, V37F, G44E, L45R, W47G, |
| Graft(IGHV3-7*01) | R97A |
| SCR-7783-001-H4 | E1D, T28P, F29S, V37F, G44E, L45R, W47G, |
| Graft(IGHV3-7*01) | K75L, N76S, R97A |
| SCR-7783-001-H5 | E1D, T28P, F29S, V37F, G44E, L45R, W47G, |
| Graft(IGHV3-7*01) | K75L, N76S, Y79W, R97A |
| SCR-7783-001-H6 | E1D, V5Q, E6A, T28P, F29S, V37F, G44E, L45R, |
| Graft(IGHV3-7*01) | W47G, K75L, N76S, R97A |
| SCR-7783-001-H7 | E1D, T28P, F29S, V37F, G44E, L45R, W47G, |
| Graft(IGHV3-7*01) | K75L, N76S, V92M, R97A, M118Q |

Note:
Graft(IGHV3-7*01) denotes that the CDRs of the nanobody are grafted into the human germline FR sequences; V37F denotes that V at position 37 of Graft(IGHV3-7*01) is mutated to F, and so on for others.

TABLE 12
Amino acid sequences of SCR-7783 humanized antibodies after back mutation

| Antibody name | Sequence No. | Amino acid sequence |
|---|---|---|
| SCR-7783-001-H1 | SEQ ID NO. 78 | EVQLVESGGGLVQPGGSLRLSCAASGFTFSRGTMAWFRQA |
| | | PGKEREGVAFINSDGRTSYAESVKGRFTISRDNAKNSLYLQ |
| | | MNSLRAEDTAVYYCAASLLYLMTVNAAAYPYWGQGTMV |
| | | TVSS |
| SCR-7783-001-H2 | SEQ ID NO. 79 | EVQLVESGGGLVQPGGSLRLSCAASGFPSSRGTMAWFRQA |
| | | PGKEREGVAFINSDGRTSYAESVKGRFTISRDNAKNSLYLQ |
| | | MNSLRAEDTAVYYCAASLLYLMTVNAAAYPYWGQGTMV |
| | | TVSS |
| SCR-7783-001-H3 | SEQ ID NO. 80 | DVQLVESGGGLVQPGGSLRLSCAASGFPSSRGTMAWFRQA |
| | | PGKEREGVAFINSDGRTSYAESVKGRFTISRDNAKNSLYLQ |
| | | MNSLRAEDTAVYYCAASLLYLMTVNAAAYPYWGQGTMV |
| | | TVSS |
| SCR-7783-001-H4 | SEQ ID NO. 81 | DVQLVESGGGLVQPGGSLRLSCAASGFPSSRGTMAWFRQA |
| | | PGKEREGVAFINSDGRTSYAESVKGRFTISRDNALSSLYLQM |
| | | NSLRAEDTAVYYCAASLLYLMTVNAAAYPYWGQGTMVTV |
| | | SS |
| SCR-7783-001-H5 | SEQ ID NO. 82 | DVQLVESGGGLVQPGGSLRLSCAASGFPSSRGTMAWFRQA |
| | | PGKEREGVAFINSDGRTSYAESVKGRFTISRDNALSSLWLQ |
| | | MNSLRAEDTAVYYCAASLLYLMTVNAAAYPYWGQGTMV |
| | | TVSS |
| SCR-7783-001-H6 | SEQ ID NO. 83 | DVQLQASGGGLVQPGGSLRLSCAASGFPSSRGTMAWFRQA |
| | | PGKEREGVAFINSDGRTSYAESVKGRFTISRDNALSSLYLQM |
| | | NSLRAEDTAVYYCAASLLYLMTVNAAAYPYWGQGTMVTV |
| | | SS |
| SCR-7783-001-H7 | SEQ ID NO. 84 | DVQLVESGGGLVQPGGSLRLSCAASGFPSSRGTMAWFRQA |
| | | PGKEREGVAFINSDGRTSYAESVKGRFTISRDNALSSLYLQM |
| | | NSLRAEDTAMYYCAASLLYLMTVNAAAYPYWGQGTQVT |
| | | VSS |

### 4.5. Back Mutation Design for SCR-7784 Humanized Antibodies

Key amino acids in the FR sequence of the SCR-7784 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 13-14.

TABLE 13
Mutation point of back mutation of SCR-7784 humanized antibody

| Antibody name | Mutation point |
|---|---|
| SCR-7784-001-H1 | H35A, V37F, G44E, L45R, W47V |
| Graft(IGHV3-30*02) | |
| SCR-7784-001-H2 | H35A, V37F, G44E, L45R, W47V, R72H |
| Graft(IGHV3-30*02) | |
| SCR-7784-001-H3 | E1D, H35A, V37F, G44E, L45R, W47V, R72H |
| Graft(IGHV3-30*02) | |
| SCR-7784-001-H4 | E1D, H35A, V37F, G44E, L45R, W47V, R72H, |
| Graft(IGHV3-30*02) | S75A |
| SCR-7784-001-H5 | E1D, H35A, V37F, G44E, L45R, W47V, F68L, |
| Graft(IGHV3-30*02) | R72H, S75A |
| SCR-7784-001-H6 | E1D, V5Q, E6A, H35A, V37F, G44E, L45R, |
| Graft(IGHV3-30*02) | W47V, R72H |
| SCR-7784-001-H7 | E1D, H35A, V37F, G44E, L45R, W47V, R72H, |
| Graft(IGHV3-30*02) | V93M, M119Q |

Note:
Graft(IGHV3-30*02) denotes that the CDRs of the nanobody are grafted into the human germline FR sequences; H35A denotes that H at position 35 of Graft(IGHV3-30*02) is mutated to A, and so on for others.

TABLE 14
Amino acid sequences of SCR-7784 humanized antibodies after back mutation

| Antibody name | Sequence No. | Amino acid sequence |
|---|---|---|
| SCR-7784-001- | SEQ ID NO. 85 | EVQLVESGGGVVQPGGSLRLSCAASGYTSNRYSMAWFRQAP |
| H1 | | GKEREVVAFIWTDGGRTYYADSVKGRFTISRDNSKNTLYLQM |
| | | NSLRAEDTAVYYCAASSLLLQTINPRAWPYWGQGTMVTVSS |
| SCR-7784-001- | SEQ ID NO. 86 | EVQLVESGGGVVQPGGSLRLSCAASGYTSNRYSMAWFRQAP |
| H2 | | GKEREVVAFIWTDGGRTYYADSVKGRFTISHDNSKNTLYLQM |
| | | NSLRAEDTAVYYCAASSLLLQTINPRAWPYWGQGTMVTVSS |
| SCR-7784-001- | SEQ ID NO. 87 | DVQLVESGGGVVQPGGSLRLSCAASGYTSNRYSMAWFRQAP |
| H3 | | GKEREVVAFIWTDGGRTYYADSVKGRFTISHDNSKNTLYLQM |
| | | NSLRAEDTAVYYCAASSLLLQTINPRAWPYWGQGTMVTVSS |
| SCR-7784-001- | SEQ ID NO. 88 | DVQLVESGGGVVQPGGSLRLSCAASGYTSNRYSMAWFRQAP |
| H4 | | GKEREVVAFIWTDGGRTYYADSVKGRFTISHDNAKNTLYLQM |
| | | NSLRAEDTAVYYCAASSLLLQTINPRAWPYWGQGTMVTVSS |
| SCR-7784-001- | SEQ ID NO. 89 | DVQLVESGGGVVQPGGSLRLSCAASGYTSNRYSMAWFRQAP |
| H5 | | GKEREVVAFIWTDGGRTYYADSVKGRLTISHDNAKNTLYLQM |
| | | NSLRAEDTAVYYCAASSLLLQTINPRAWPYWGQGTMVTVSS |
| SCR-7784-001- | SEQ ID NO. 90 | DVQLQASGGGVVQPGGSLRLSCAASGYTSNRYSMAWFRQAP |
| H6 | | GKEREVVAFIWTDGGRTYYADSVKGRFTISHDNSKNTLYLQM |
| | | NSLRAEDTAVYYCAASSLLLQTINPRAWPYWGQGTMVTVSS |
| SCR-7784-001- | SEQ ID NO. 91 | DVQLVESGGGVVQPGGSLRLSCAASGYTSNRYSMAWFRQAP |
| H7 | | GKEREVVAFIWTDGGRTYYADSVKGRFTISHDNSKNTLYLQM |
| | | NSLRAEDTAMYYCAASSLLLQTINPRAWPYWGQGTQVTVSS |

### 4.6. Back Mutation Design for SCR-7785 Humanized Antibodies

The CDR sequences of the SCR-7785 antibody were defined based on the Kabat scheme. Key amino acids in the FR sequence of the SCR-7785 humanized antibody were back-mutated to amino acids corresponding to the camelid antibody to ensure the original affinity. Details of the mutation points of the specific back mutation (back mutation points were numbered in a natural order) and the specific amino acid sequences are shown in Tables 15-16.

TABLE 15
Mutation point of back mutation of SCR-7785 humanized antibody

| Antibody name | Mutation point |
|---|---|
| SCR-7785-001-H1 | V37F, G44E, L45R, W47A, K97A |
| Graft(IGHV3-30*02) | |
| SCR-7785-001-H2 | V37F, G44E, L45R, W47A, R71Q, K97A |
| Graft(IGHV3-30*02) | |
| SCR-7785-001-H3 | F27D, V37F, G44E, L45R, W47A, R71Q, K97A |
| Graft(IGHV3-30*02) | |
| SCR-7785-001-H4 | F27D, V37F, G44E, L45R, W47A, R71Q, S74T, |
| Graft(IGHV3-30*02) | K97A |
| SCR-7785-001-H5 | F27D, V37F, G44E, L45R, W47A, R71Q, |
| Graft(IGHV3-30*02) | V92M,K97A, M118Q |
| SCR-7785-001-H6 | F27D, V37F, A40R, G44E, L45R, W47A, R71Q, |
| Graft(IGHV3-30*02) | V92M, K97A, M118Q |
| SCR-7785-001-H7 | V5Q, E6A, F27D, V37F, G44E, L45R, W47A, |
| Graft(IGHV3-30*02) | R71Q, K97A |

Note:
Graft(IGHV3-30*02) denotes that the CDRs of the camelid antibody are grafted into the human germline FR sequences; V37F denotes that V at position 37 of Graft(IGHV3-30*02) is mutated to F, and so on for others.

TABLE 16
Amino acid sequences of SCR-7785 humanized antibodies after back mutation

| Antibody name | Sequence No. | Amino acid sequence |
|---|---|---|
| SCR-7785-001- | SEQ ID NO. 92 | EVQLVESGGGVVQPGGSLRLSCAASGFTFSRGSWGWFRQAPG |
| H1 | | KEREAVALISSDGWTFYADSVKGRFTISRDNSKNTLYLQMNSL |
| | | RAEDTAVYYCAASVLVLRTLNPKAWHHWGQGTMVTVSS |
| SCR-7785-001- | SEQ ID NO. 93 | EVQLVESGGGVVQPGGSLRLSCAASGFTFSRGSWGWFRQAPG |
| H2 | | KEREAVALISSDGWTFYADSVKGRFTISQDNSKNTLYLQMNSL |
| | | RAEDTAVYYCAASVLVLRTLNPKAWHHWGQGTMVTVSS |
| SCR-7785-001- | SEQ ID NO. 94 | EVQLVESGGGVVQPGGSLRLSCAASGDTFSRGSWGWFRQAP |
| H3 | | GKEREAVALISSDGWTFYADSVKGRFTISQDNSKNTLYLQMNS |
| | | LRAEDTAVYYCAASVLVLRTLNPKAWHHWGQGTMVTVSS |
| SCR-7785-001- | SEQ ID NO. 95 | EVQLVESGGGVVQPGGSLRLSCAASGDTFSRGSWGWFRQAP |
| H4 | | GKEREAVALISSDGWTFYADSVKGRFTISQDNTKNTLYLQMNS |
| | | LRAEDTAVYYCAASVLVLRTLNPKAWHHWGQGTMVTVSS |
| SCR-7785-001- | SEQ ID NO. 96 | EVQLVESGGGVVQPGGSLRLSCAASGDTFSRGSWGWFRQAP |
| H5 | | GKEREAVALISSDGWTFYADSVKGRFTISQDNSKNTLYLQMNS |
| | | LRAEDTAMYYCAASVLVLRTLNPKAWHHWGQGTQVTVSS |
| SCR-7785-001- | SEQ ID NO. 97 | EVQLVESGGGVVQPGGSLRLSCAASGDTFSRGSWGWFRQRP |
| H6 | | GKEREAVALISSDGWTFYADSVKGRFTISQDNSKNTLYLQMNS |
| | | LRAEDTAMYYCAASVLVLRTLNPKAWHHWGQGTQVTVSS |
| SCR-7785-001- | SEQ ID NO. 98 | EVQLQASGGGVVQPGGSLRLSCAASGDTFSRGSWGWFRQAP |
| H7 | | GKEREAVALISSDGWTFYADSVKGRFTISQDNSKNTLYLQMNS |
| | | LRAEDTAVYYCAASVLVLRTLNPKAWHHWGQGTMVTVSS |

### Example 5. Expression, Purification, and Identification of Humanized Nanobody

### 5.1 Expression and Purification of Humanized Nanobody

The humanized antibody variable region sequence gene was synthesized and cloned into a pTT5 vector with a human hinge region and an Fc constant region sequence to form a VHH-huFc (C220S) expression order, and a plasmid was prepared. The antibody plasmid was transiently transfected into Expi293F cells by PEI, and after 7 days of culture, the supernatant was collected, and the antibody was prepared by protein A purification as in Example 1.3.2.

### 5.2 Assay on Binding of Humanized Antibodies to Human MUC17-his Protein by Enzyme-Linked Immunosorbent Assay (ELISA)

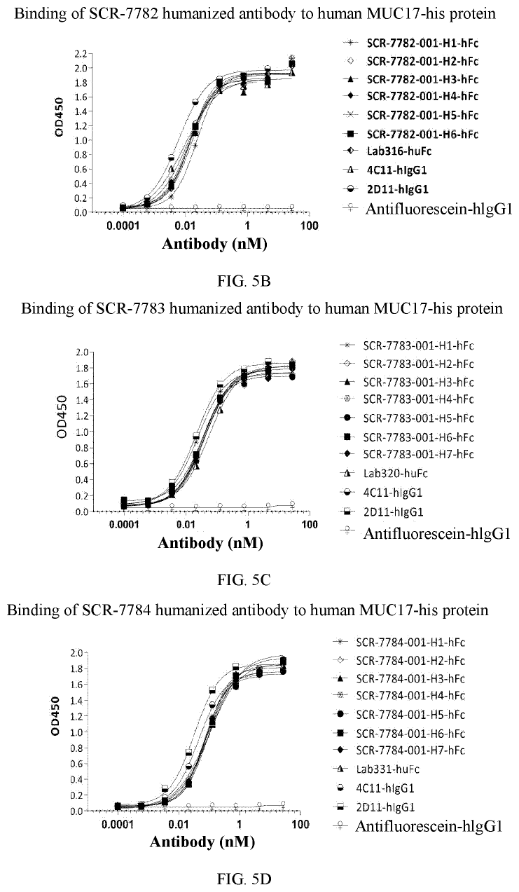

See the method and steps in Example 3.2 for the assay on the binding activity of the humanized antibody to the human MUC17-his protein. The experimental results are shown in FIGS. 5A-5E. The humanized antibodies SCR-7724, SCR-7782, SCR-7783, SCR-7784, and SCR-7785 all maintain the binding ability of the recombinant antibody and have good binding activity with the human MUC17 protein.

5.3 Assay on Binding Activity of Humanized Antibody with Monkey MUC17-his Protein by ELISA

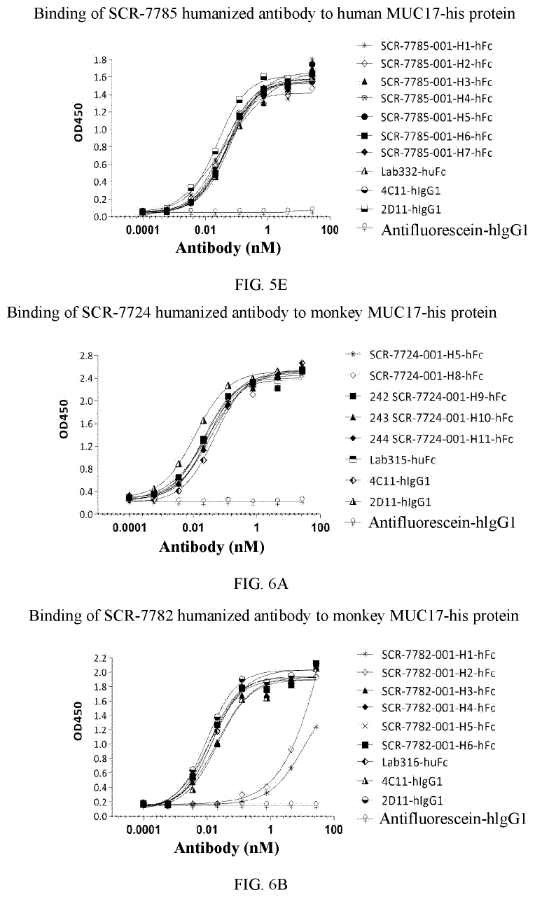

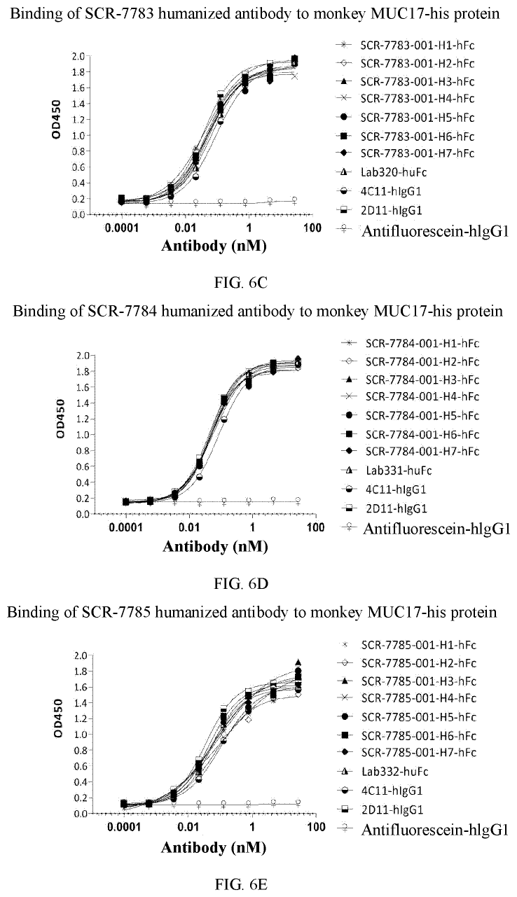

To assay the binding activity of the humanized antibodies with the monkey MUC17-his protein, the monkey MUC17-his protein was diluted with PBS to a final concentration of 2 g/mL and subjected to ELISA assay and data analysis according to the method described in Example 3.3. The results are shown in FIGS. 6A-6E. The humanized antibodies SCR-7724, SCR-7782, SCR-7783, SCR-7784, and SCR-7785 have cross-binding activity with the monkey MUC17 protein, maintaining the binding ability of the recombinant antibody.

### 5.4 Assay on Binding of Humanized Antibodies to Endogenous Tumor Cells NUGC4 by Flow Cytometry Assay (FACS)

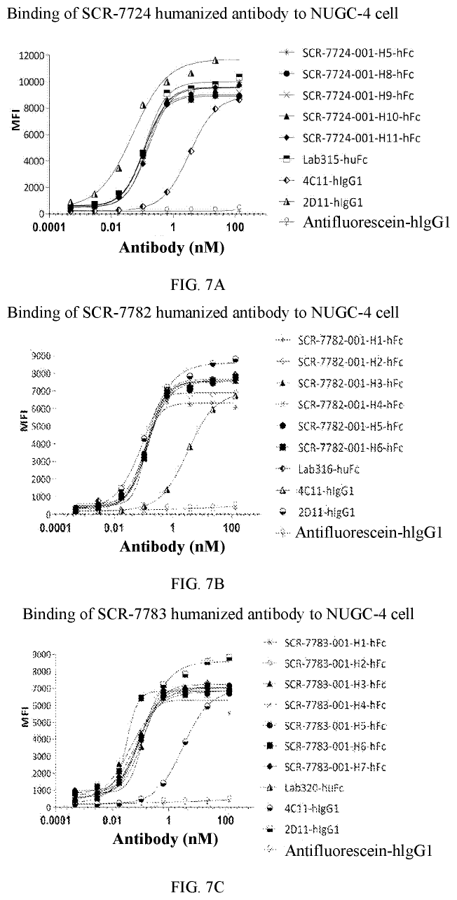

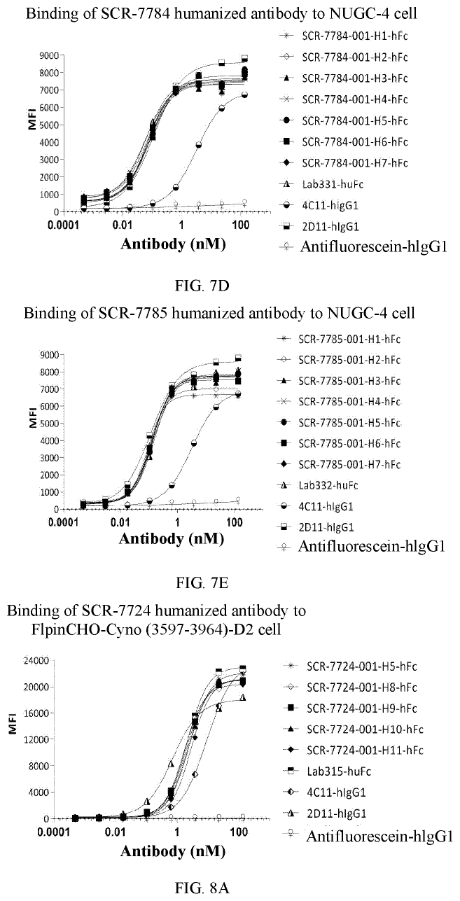

The preparation of the assay cells and the antibodies to be tested and the assay were performed with reference to Example 3.4. The assay results are shown in FIGS. 7A-7E. The humanized antibodies all have a relatively good binding activity with endogenous cells NUGC4, maintaining the binding ability of the corresponding recombinant antibodies before humanization.

### 5.5 Assay on Binding of Humanized Antibodies to Monkey MUC17 Overexpression Cells by Flow Cytometry Assay (FACS)

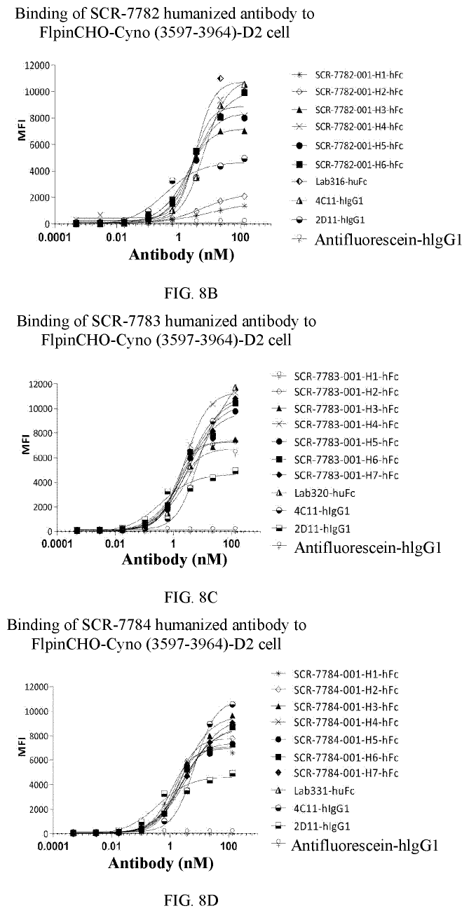

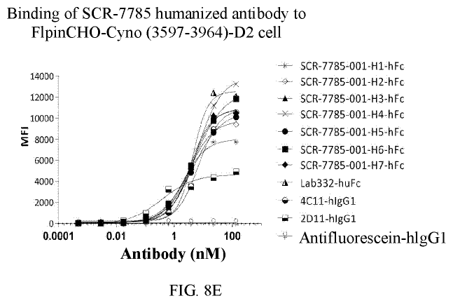

Preparation of monkey MUC17 overexpression cells FlpinCHO-Cyno(3597-3964)-D2 and the FACS assay method were referring to Examples 3.4 and 3.5. The experimental results are shown in FIGS. 8A-8E. The humanized antibodies SCR-7724, SCR-7782, SCR-7783, SCR-7784, and SCR-7785 in this example all have a relatively good cross-binding activity with the recombinant cells FlpinCHO-Cyno(3597-3964)-D2, maintaining the equivalent binding ability of the recombinant antibody.

### Example 6. Assay on Affinity of Humanized Antibodies

After the finally obtained various humanized antibodies were expressed and purified, the affinity of the humanized antibodies for the human MUC17 protein was assayed according to the method in Example 3.6. The specific affinity values are shown in Table 17.

TABLE 17
Assay on affinity of humanized antibodies
for human MUC17 protein by SPR (biacore)

Antigen name
Human MUC17-ECD4131-His
Binding kinetics

| Antibody name | ka(1/Ms) | kd(1/s) | KD(M) |
|---|---|---|---|
| Lab315-huFc | 2.37E+05 | 2.90E−05 | 1.23E−10 |
| SCR-7724-001-H5-hFc | 1.31E+05 | 4.02E−05 | 3.06E−10 |
| SCR-7724-001-H8-hFc | 7.24E+04 | 1.70E−03 | 2.35E−08 |
| SCR-7724-001-H9-hFc | 9.56E+04 | 5.83E−04 | 6.10E−09 |
| SCR-7724-001-H10-hFc | 1.05E+05 | 8.58E−05 | 8.15E−10 |
| SCR-7724-001-H11-hFc | 1.09E+05 | 7.63E−05 | 6.97E−10 |
| Lab316-huFc | 2.73E+05 | 3.28E−05 | 1.20E−10 |
| SCR-7782-001-H1-hFc | 1.86E+05 | 4.11E−04 | 2.21E−09 |
| SCR-7782-001-H2-hFc | 2.00E+05 | 2.56E−04 | 1.28E−09 |
| SCR-7782-001-H3-hFc | 1.92E+05 | 2.58E−05 | 1.34E−10 |
| SCR-7782-001-H4-hFc | 2.24E+05 | 1.32E−05 | 5.87E−11 |
| SCR-7782-001-H5-hFc | 1.99E+05 | 9.72E−06 | 4.90E−11 |
| SCR-7782-001-H6-hFc | 2.31E+05 | 1.23E−05 | 5.30E−11 |
| Lab320-huFc | 1.96E+05 | 1.03E−04 | 5.27E−10 |
| SCR-7783-001-H1-hFc | 1.45E+05 | 1.76E−04 | 1.21E−09 |
| SCR-7783-001-H2-hFc | 2.10E+05 | 8.28E−05 | 3.94E−10 |
| SCR-7783-001-H3-hFc | 2.10E+05 | 8.54E−05 | 4.07E−10 |
| SCR-7783-001-H4-hFc | 2.14E+05 | 8.77E−05 | 4.09E−10 |
| SCR-7783-001-H5-hFc | 1.88E+05 | 9.07E−05 | 4.83E−10 |
| SCR-7783-001-H6-hFc | 1.93E+05 | 9.61E−05 | 4.98E−10 |
| SCR-7783-001-H7-hFc | 2.18E+05 | 8.52E−05 | 3.91E−10 |
| Lab331-huFc | 7.26E+05 | 6.39E−05 | 8.80E−11 |
| SCR-7784-001-H1-hFc | 3.01E+05 | 2.27E−05 | 7.55E−11 |
| SCR-7784-001-H2-hFc | 3.68E+05 | 2.77E−05 | 7.53E−11 |
| SCR-7784-001-H3-hFc | 3.69E+05 | 2.69E−05 | 7.30E−11 |
| SCR-7784-001-H4-hFc | 3.72E+05 | 2.69E−05 | 7.22E−11 |
| SCR-7784-001-H5-hFc | 3.92E+05 | 3.93E−05 | 1.00E−10 |
| SCR-7784-001-H6-hFc | 3.13E+05 | 3.24E−05 | 1.04E−10 |
| SCR-7784-001-H7-hFc | 3.67E+05 | 3.05E−05 | 8.31E−11 |
| Lab332-huFc | 5.04E+05 | 1.19E−04 | 2.36E−10 |
| SCR-7785-001-H1-hFc | 2.91E+05 | 4.55E−04 | 1.56E−09 |
| SCR-7785-001-H2-hFc | 3.63E+05 | 6.90E−04 | 1.90E−09 |
| SCR-7785-001-H3-hFc | 3.09E+05 | 7.24E−05 | 2.34E−10 |
| SCR-7785-001-H4-hFc | 3.07E+05 | 7.41E−05 | 2.41E−10 |
| SCR-7785-001-H5-hFc | 3.00E+05 | 7.19E−05 | 2.40E−10 |
| SCR-7785-001-H6-hFc | 2.84E+05 | 6.29E−05 | 2.22E−10 |
| SCR-7785-001-H7-hFc | 2.90E+05 | 8.41E−05 | 2.90E−10 |

Although the technical solutions of the present application have been described in detail herein above through general explanations and specific embodiments, it will be apparent to those skilled in the art that modifications or improvements can be made based on the technical solutions. Accordingly, such modifications and improvements made without departing from the spirit of the present application all fall within the protection scope of the present application.

1. A nanobody or an antigen-binding fragment thereof for specifically binding to MUC17, wherein the nanobody or the antigen-binding fragment thereof comprises a CDR1, a CDR2, and a CDR3; the CDR1, the CDR2, and the CDR3 are respectively selected from a CDR1, a CDR2, and a CDR3 of the VHH set forth in any one of SEQ ID NOs: 8-12, 62-66, and 67-98.

2. The nanobody or the antigen-binding fragment thereof according to claim 1, wherein the CDR1, the CDR2, and the CDR3 are determined according to the Kabat numbering scheme, the Chothia numbering scheme, or the IMGT numbering scheme; for example, the CDR1 comprises the amino acid sequence set forth in SEQ ID NO: 13, 16, 19, 22, 25, 28, 31, 34, 37, 40, 43, 46, 49, 52, or 55; the CDR2 comprises the amino acid sequence set forth in SEQ ID NO: 14, 17, 20, 23, 26, 29, 32, 35, 38, 41, 44, 47, 50, 53, or 56; the CDR3 comprises the amino acid sequence set forth in SEQ ID NO: 15, 18, 21, 24, 27, 30, 33, 36, 39, 42, 45, 48, 51, 54, or 57.

3. The nanobody or the antigen-binding fragment thereof according to claim 1, wherein CDR1s, CDR2s, and CDR3s of the VHHs set forth in SEQ ID NOs: 8, 62, and 67-71 are according to the IMGT, Kabat, or Chothia numbering scheme, and have the amino acid sequences set forth in SEQ ID NOs: 13-15, SEQ ID NOs: 28-30, or SEQ ID NOs: 43-45;
CDR1-3s of the VHHs set forth in SEQ ID NOs: 9, 63, and 72-77 are according to the IMGT, Kabat, or Chothia numbering scheme, and have the amino acid sequences set forth in SEQ ID NOs: 16-18, SEQ ID NOs: 31-33, or SEQ ID NOs: 46-48;
CDR1-3s of the VHHs set forth in SEQ ID NOs: 10, 64, and 78-84 are according to the IMGT, Kabat, or Chothia numbering scheme, and have the amino acid sequences set forth in SEQ ID NOs: 19-21, SEQ ID NOs: 34-36, or SEQ ID NOs: 49-51;
CDR1-3s of the VHHs set forth in SEQ ID NOs: 11, 65, and 85-91 are according to the IMGT, Kabat, or Chothia numbering scheme, and have the amino acid sequences set forth in SEQ ID NOs: 22-24, SEQ ID NOs: 37-39, or SEQ ID NOs: 52-54;
CDR1-3s of the VHHs set forth in SEQ ID NOs: 12, 66, and 92-98 are according to the IMGT, Kabat, or Chothia numbering scheme, and have the amino acid sequences set forth in SEQ ID NOs: 25-27, SEQ ID NOs: 40-42, or SEQ ID NOs: 55-57.

4. The nanobody or the antigen-binding fragment thereof according to claim 1, wherein the nanobody or the antigen-binding fragment thereof comprises CDR1, CDR2, and CDR3 sequences having at least 80%, 85%, 90%, 91%, 92%, 93%, 94%, 95%, 96%, 97%, 98%, 99%, or 100% identity to the CDR1, the CDR2, and the CDR3 or having 1, 2, 3, or more amino acid insertions, deletions, and/or substitutions compared with the CDR1, the CDR2, and the CDR3, and preferably, the substitutions are conservative amino acid substitutions.

5. The nanobody or the antigen-binding fragment thereof according to claim 1, wherein the nanobody or the antigen-binding fragment thereof comprises the VHH set forth in any one of SEQ ID NOs: 8-12, 62-66, and 67-98, or a VHH sequence having at least 80%, 85%, 90%, 91%, 92%, 93%, 94%, 95%, 96%, 97%, 98%, 99%, or 100% identity to the VHH set forth in any one of SEQ ID NOs: 8-12, 62-66, and 67-98 or at most 20, 19, 18, 17, 16, 15, 14, 13, 12, 11, 10, 9, 8, 7, 6, 5, 4, 3, 2, or 1 mutation compared with the VHH set forth in any one of SEQ ID NOs: 8-12, 62-66, and 67-98; the mutation is selected from an insertion, a deletion, and/or a substitution, and preferably, the substitution is a conservative amino acid substitution.

6. The nanobody or the antigen-binding fragment thereof according to claim 5, wherein the nanobody or the antigen-binding fragment thereof comprises a framework region sequence at least having a mutation, compared with a framework region of the VHH set forth in SEQ ID NO: 62, selected from the group consisting of: numbered in the natural order, H34G, V36F, G43E, L44R, W46G, S48A, G57I, R70K, Y93S, W114R, or M119Q; preferably, at least having H34G, V36F, G43E, L44R, W46G, S48A, G57I, R70K, Y93S, and W 114R mutations; more preferably, at least having H34G, V36F, G43E, L44R, W46G, S48A, and W114R mutations; more preferably, at least having H34G, V36F, G43E, L44R, W46G, S48A, Y93S, and W114R mutations; more preferably, at least having H34G, V36F, G43E, L44R, W46G, S48A, R70K, Y93S, and W114R mutations; more preferably, at least having H34G, V36F, G43E, L44R, W46G, S48A, R70K, Y93S, W114R, and M119Q mutations;
the antibody or the antigen-binding fragment thereof comprises a framework region sequence at least having a mutation, compared with a framework region of the VHH set forth in SEQ ID NO: 63, selected from the group consisting of: numbered in the natural order, E1D, V5Q, E6A, G26D, F27N, V37F, G44E, L45R, W47G, R71Q, V92M, R97A, or M118Q; preferably, at least having V37F, G44E, L45R, W47G, and R97A mutations; more preferably, at least having V37F, G44E, L45R, W47G, R71Q, and R97 mutations; more preferably, at least having F27N, V37F, G44E, L45R, W47G, R71Q, and R97A mutations; more preferably, at least having E1D, G26D, F27N, V37F, G44E, L45R, W47G, R71Q, and R97A mutations; more preferably, at least having E1D, V5Q, E6A, G26D, F27N, V37F, G44E, L45R, W47G, R71Q, and R97A mutations; more preferably, at least having E1D, G26D, F27N, V37F, G44E, L45R, W47G, R71Q, V92M, R97A, and M118Q mutations;
the antibody or the antigen-binding fragment thereof comprises a framework region sequence at least having a mutation, compared with a framework region of the VHH set forth in SEQ ID NO: 64, selected from the group consisting of: numbered in the natural order, E1D, V5Q, E6A, T28P, F29S, V37F, G44E, L45R, W47G, K75L, N76S, Y79W, V92M, R97A, or M118Q; preferably, at least having V37F, G44E, L45R, W47G, and R97A mutations; more preferably, at least having T28P, F29S, V37F, G44E, L45R, W47G, and R97A mutations; more preferably, at least having E1D, T28P, F29S, V37F, G44E, L45R, W47G, and R97A mutations; more preferably, at least having E1D, T28P, F29S, V37F, G44E, L45R, W47G, K75L, N76S, and R97A mutations; more preferably, at least having E1D, T28P, F29S, V37F, G44E, L45R, W47G, K75L, N76S, Y79W, and R97A mutations; more preferably, at least having E1D, V5Q, E6A, T28P, F29S, V37F, G44E, L45R, W47G, K75L, N76S, and R97A mutations; more preferably, at least having E1D, T28P, F29S, V37F, G44E, L45R, W47G, K75L, N76S, V92M, R97A, and M118Q mutations;
the antibody or the antigen-binding fragment thereof comprises a framework region sequence at least having a mutation, compared with a framework region of the VHH set forth in SEQ ID NO: 65, selected from the group consisting of: numbered in the natural order, E1D, V5Q, E6A, H35A, V37F, G44E, L45R, W47V, F68L, R72H, S75A, V93M, or M119Q; preferably, at least having H35A, V37F, G44E, L45R, and W47V mutations; more preferably, at least having H35A, V37F, G44E, L45R, W47V, and R72H mutations; more preferably, at least having E1D, H35A, V37F, G44E, L45R, W47V, and R72H mutations; more preferably, at least having E1D, H35A, V37F, G44E, L45R, W47V, R72H, and S75A mutations; more preferably, at least having E1D, H35A, V37F, G44E, L45R, W47V, F68L, R72H, and S75A mutations; more preferably, at least having E1D, V5Q, E6A, H35A, V37F, G44E, L45R, W47V, and R72H mutations; more preferably, at least having E1D, H35A, V37F, G44E, L45R, W47V, R72H, V93M, and M119Q mutations;
or the antibody or the antigen-binding fragment thereof comprises a framework region sequence at least having a mutation, compared with a framework region of the VHH set forth in SEQ ID NO: 66, selected from the group consisting of: numbered in the natural order, V5Q, E6A, F27D, V37F, A40R, G44E, L45R, W47A, R71Q, S74T, V92M, K97A, or M118Q; preferably, at least having V37F, G44E, L45R, W47A, and K97A mutations; more preferably, at least having V37F, G44E, L45R, W47A, R71Q, and K97A mutations; more preferably, at least having F27D, V37F, G44E, L45R, W47A, R71Q, and K97A mutations; more preferably, at least having F27D, V37F, G44E, L45R, W47A, R71Q, S74T, and K97A mutations; more preferably, at least having F27D, V37F, G44E, L45R, W47A, R71Q, V92M, K97A, and M118Q mutations; more preferably, at least having F27D, V37F, A40R, G44E, L45R, W47A, R71Q, V92M, K97A, and M118Q mutations; more preferably, at least having V5Q, E6A, F27D, V37F, G44E, L45R, W47A, R71Q, and K97A mutations.

7. The nanobody or the antigen-binding fragment thereof according to claim 1, wherein the nanobody or the antigen-binding fragment thereof specifically binds to a human MUC17 protein and/or a monkey MUC17 protein; preferably, the nanobody or the antigen-binding fragment thereof binds to human MUC17 protein and/or monkey MUC17 protein with a KD superior to 1.00E−7 M: optionally, wherein the nanobody or the antigen-binding fragment thereof is: (1) a chimeric antibody or a fragment thereof, (2) a humanized antibody or a fragment thereof, or (3) a fully human antibody or a fragment thereof, optionally, wherein the nanobody or the antigen-binding fragment thereof comprises or does not comprise an antibody heavy chain constant region: optionally, the antibody heavy chain constant region is selected from human, Vicugna pacos, mouse, rat, rabbit, or sheep: optionally, the antibody heavy chain constant region is selected from IgG, IgM, IgA, IgE, or IgD, and the IgG is selected from IgG1, IgG2, IgG3, or IgG4; optionally, the heavy chain constant region is selected from an Fc region, a CH3 region, or an intact heavy chain constant region: preferably, the heavy chain constant region is a human Fc region: preferably, the nanobody or the antigen-binding fragment thereof is a heavy chain antibody, optionally, wherein the nanobody or the antigen-binding fragment thereof is further conjugated to a therapeutic agent or a tracer: preferably, the therapeutic agent is selected from a drug, a toxin, a radioisotope, a chemotherapeutic agent, or an immunomodulator, and the tracer is selected from a radiocontrast medium, a paramagnetic ion, a metal, a fluorescent label, a chemiluminescent label, an ultrasound contrast agent, and a photosensitizer.

8. (canceled)

9. (canceled)

10. (canceled)

11. A multispecific molecule, wherein the multispecific molecule comprises the nanobody or the antigen-binding fragment thereof according to claim 1; preferably, the multispecific molecule further comprises a nanobody or an antigen-binding fragment thereof specifically binding to an antigen other than MUC17 or binding to a MUC17 epitope different from that of the nanobody or the antigen-binding fragment thereof; optionally, wherein the antigen other than MUC17 is an antigen on the surface of a T cell, a B cell, a natural killer cell, a dendritic cell, a macrophage, a monocyte, or a neutrophil; preferably, the antigen other than MUC17 is selected from: CD96, PD-1, PD-L1, PD-L2, OX40, OX40L, LAG-3, TIM3, VISTA, CD3, CD3y, CD36, CD3g, CD3, CD27, CD28, CD28H, CD16, CD16A, CD32B, VEGF, NKG2D, NKp30, NKp46, NKp44, CD19, CD20, CD40, CD47, 4-1BB, ICOS, OX40, EGFR, EGFRvIII, TNF-alpha, CD33, HER2, HER3, HAS, CD5, CD27, EphA2, EpCAM, MUC1, MUC16, CEA, Claudin18.2, a folate receptor, Claudin6, WT1, NY-ESO-1, MAGE3, ASGPR1, TGFβ-trap, IL-2, IL-15, IL-21, IL-18, or CDH16; preferably, the multispecific molecule is bispecific, trispecific, or tetraspecific, and more preferably, the multispecific molecule is divalent, tetravalent, or hexavalent, optionally, wherein the multispecific molecule is a tandem scFv, a bifunctional antibody (Db), a single chain bifunctional antibody (scDb), a dual affinity retargeting (DART) antibody, a F(ab′)2, a dual variable domain (DVD) antibody, a knobs-into-holes (KiH) antibody, a dock-and-lock (DNL) antibody, a chemically cross-linked antibody, a heteropolymeric nanobody, or a heteroconjugate antibody.

12. (canceled)

13. (canceled)

14. A chimeric antigen receptor (CAR), wherein the chimeric antigen receptor at least comprises an extracellular antigen-binding domain, a transmembrane domain, and an intracellular signaling domain; the extracellular antigen-binding domain comprises the nanobody or the antigen-binding fragment thereof according to claim 1.

15. An immune effector cell, wherein the immune effector cell expresses or comprises a nucleic acid fragment encoding the chimeric antigen receptor according to claim 14; preferably, the immune effector cell is selected from a T cell, a natural killer cell (NK cell), a natural killer T cell (NKT cell), a double negative T cell (DNT cell), a monocyte, a macrophage, a dendritic cell, or a mast cell, and the T cell is preferably selected from a cytotoxic T cell, a regulatory T cell, or a helper T cell; preferably, the immune effector cell is an auto-immune effector cell or an allogeneic immune effector cell.

16. An isolated nucleic acid fragment, wherein the nucleic acid fragment encodes the nanobody or the antigen-binding fragment thereof according to claim 1.

17. A vector, wherein the vector comprises the isolated nucleic acid fragment according to claim 16.

18. A host cell, wherein the host cell comprises the vector according to claim 17; preferably, the cell is a prokaryotic cell or a eukaryotic cell, such as a bacterium (E. coli), a fungus (yeast), an insect cell, or a mammalian cell (a CHO cell line or a 293T cell line).

19. A method for preparing the nanobody or the antigen-binding fragment thereof according to, wherein the method comprises culturing the cell according to claim 18; and isolating a nanobody or an antigen-binding fragment thereof expressed by the cell, or isolating a multispecific molecule expressed by the cell.

20. A method for preparing the immune effector cell according to claim 15, wherein the method comprises introducing a nucleic acid fragment encoding the CAR into the immune effector cell; optionally, the method further comprises initiating expression of the CAR in the immune effector cell.

21. A pharmaceutical composition, wherein the pharmaceutical composition comprises the nanobody or the antigen-binding fragment thereof according to claim 1; optionally, the pharmaceutical composition further comprises a pharmaceutically acceptable carrier, diluent, or adjuvant.

22. (canceled)

23. A method for preventing and/or treating a tumor, wherein the method comprises administering to a patient in need thereof an effective amount of the nanobody or the antigen-binding fragment thereof according to claim 1, wherein optionally, the tumor is a tumor that expresses MUC17 on the cell surface, for example, gastric cancer, pancreatic cancer, and gastroesophageal junction carcinoma.

24. (canceled)

25. A kit, wherein the kit comprises the nanobody or the antigen-binding fragment thereof according to claim 1.

26. A method for detecting MUC17 expression, the method comprising contacting a sample to be tested with the nanobody or the antigen-binding fragment thereof according to claim 1 under a condition allowing formation of a complex by the nanobody or the antigen-binding fragment thereof according to and MUC17.

27. A method for inhibiting the proliferation or migration of a cell expressing MUC17 in vitro, the method comprising contacting the cell with the nanobody or the antigen-binding fragment thereof according to claim 1 under a condition allowing formation of a complex by the nanobody or the antigen-binding fragment thereof and MUC17.


SEQUENCE LISTING (98 sequences)

SEQ ID NO: 1 - protein, 269 aa, synthetic construct
RTTTCFGDGCQNTASRCKNGGTWDGLKCQCPNLYYGELCEEVVSSIDIGPPETISAQMELTVTVTSVKFTEELKNHSSQEFQEFKQTFTEQMNIVYSGIPEYVGVNITKLRLGSVVVEHDVLLRTKYTPEYKTVLDNATEVVKEKITKVTTQQIMINDICSDMMCFNTTGTQVQNITVTQYDPEEDCRKMAKEYGDYFVVEYRDQKPYCISPCEPGFSVSKNCNLGKCQMSLSGPQCLCVTTETHWYSGETCNQGTQKSLVYGHHHHHH

SEQ ID NO: 2 - protein, 291 aa, synthetic construct
PLATIPVSTTSLTSSEGSTISTPSVDISTPVTTSIVGDQCSRCKNGGFWDGLKCQCLTPYYGESCEEVVNSIDIAPPETVSAQMELTVTVTSVKFTDELKNHSSQEFREFNKTFTEQMNIVYSGIPEYVGVNITNLRLGSVVVEHDVLLRTKYTPEYKTALDNATEVVKQKITKVTTEQIMTNDNCSALMCFNTTGTQVQNITVTQYDPVEECRQKAEEYEDYFLVEYRDQKPYCISPCESGFNASKNCNHGKCQMSQNGARCLCVTTETHWYSGEDCNQGTQKSHHHHHH

SEQ ID NO: 3 - protein, 451 aa, synthetic construct
QVQLVESGGGVVQPGRSLRLSCAASGFTFSNHGMHWVRQAPGKCLEWVAGIWSDASNKYYAEAVKGRFTISRDTSKNTLYLQMNSLRAEDTAVYYCARATYTTGWSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
SYELTQSPSVSVSPGQTASITCSGDKLGDKYTSWYQQKPGQSPVLVIYHDRKRPSGIPERFSGSNSGNTATLTISGTQAMDEADYYCQAYDRSTAWVFGCGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, synthetic construct
QVQLVESGGGVVQPGRSLRLSCAASGFTFSSYGMHWVRQAPGKCLEWVAVISYDASNKYYASAVKGRFTISRDNSKNTLYLQMNSLRAEDTAVYYCARGAYTYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, synthetic construct
EIVLTQSPGTLSLSPGERATLSCRASQSVNRYLAWYQQKPGQAPRLLIYGASNRATGIPDRFTGSGSGTDFTLTISRLEPEDFAVYFCHHYGSSIFAFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 124 aa, synthetic construct
DVQLQASGGGAVQAGGSLRLSCAASGDITRRCMGWFRQAPGKEREGVAGISEDGITIYADSVKGRFTISKDNAKNSLYLQMNSLAPEDTANYSCAASILNRVGCRQLSYEYIYRGQGTQVTVSS

SEQ ID NO: 9 - protein, 123 aa, synthetic construct
DVQLQASGGGSVQAGGSLRLSCAASDNTFSRGSMSWFRQAPGKEREGVAIISSDGWTGYADSVKGRFTISQDNAKNTLYLQMIGLKPDDTAMYYCAASFLVLRALNPKAWHYWGQGTQVTVSS

SEQ ID NO: 10 - protein, 123 aa, synthetic construct
DVQLQASGGGSVQSGGSLRLSCAASGFPSSRGTMAWFRQAPGREREGVAFINSDGRTSYAESVKGRFTISRDNALSTLWLQMNSLKPEDTGMYYCAASLLYLMTVNAAAYPYWGQGTQVTVSS

SEQ ID NO: 11 - protein, 124 aa, synthetic construct
DVQLQASGGGSVQAGGSLRLSCAASGYTSNRYSMAWFRQAPGKEREVVAFIWTDGGRTYYADSVKGRLTISHDNAKNTLYLQMDSLKPEDTAMYYCAASSLLLQTINPRAWPYWGQGTQVTVSS

SEQ ID NO: 12 - protein, 123 aa, synthetic construct
EVQLQASGGGSVQAGGSLRVSCAASGDTFSRGSWGWFRQRPGKEREAVALISSDGWTFYADSVKGRFTISQDNTKNTLYLQMISLRPEDTAMYYCAASVLVLRTLNPKAWHHWGQGTQVTVSS

SEQ ID NO: 13 - protein, 7 aa, synthetic construct
GDITRRC

SEQ ID NO: 14 - protein, 7 aa, synthetic construct
ISEDGIT

SEQ ID NO: 15 - protein, 19 aa, synthetic construct
AASILNRVGCRQLSYEYIY

SEQ ID NO: 16 - protein, 8 aa, synthetic construct
DNTFSRGS

SEQ ID NO: 17 - protein, 7 aa, synthetic construct
ISSDGWT

SEQ ID NO: 18 - protein, 17 aa, synthetic construct
AASFLVLRALNPKAWHY

SEQ ID NO: 19 - protein, 8 aa, synthetic construct
GFPSSRGT

SEQ ID NO: 20 - protein, 7 aa, synthetic construct
INSDGRT

SEQ ID NO: 21 - protein, 17 aa, synthetic construct
AASLLYLMTVNAAAYPY

SEQ ID NO: 22 - protein, 8 aa, synthetic construct
GYTSNRYS

SEQ ID NO: 23 - protein, 8 aa, synthetic construct
IWTDGGRT

SEQ ID NO: 24 - protein, 17 aa, synthetic construct
AASSLLLQTINPRAWPY

SEQ ID NO: 25 - protein, 8 aa, synthetic construct
GDTFSRGS

SEQ ID NO: 26 - protein, 7 aa, synthetic construct
ISSDGWT

SEQ ID NO: 27 - protein, 17 aa, synthetic construct
AASVLVLRTLNPKAWHH

SEQ ID NO: 28 - protein, 4 aa, synthetic construct
RCMG

SEQ ID NO: 29 - protein, 16 aa, synthetic construct
GISEDGITIYADSVKG

SEQ ID NO: 30 - protein, 17 aa, synthetic construct
SILNRVGCRQLSYEYIY

SEQ ID NO: 31 - protein, 5 aa, synthetic construct
RGSMS

SEQ ID NO: 32 - protein, 16 aa, synthetic construct
IISSDGWTGYADSVKG

SEQ ID NO: 33 - protein, 15 aa, synthetic construct
SFLVLRALNPKAWHY

SEQ ID NO: 34 - protein, 5 aa, synthetic construct
RGTMA

SEQ ID NO: 35 - protein, 16 aa, synthetic construct
FINSDGRTSYAESVKG

SEQ ID NO: 36 - protein, 15 aa, synthetic construct
SLLYLMTVNAAAYPY

SEQ ID NO: 37 - protein, 5 aa, synthetic construct
RYSMA

SEQ ID NO: 38 - protein, 17 aa, synthetic construct
FIWTDGGRTYYADSVKG

SEQ ID NO: 39 - protein, 15 aa, synthetic construct
SSLLLQTINPRAWPY

SEQ ID NO: 40 - protein, 5 aa, synthetic construct
RGSWG

SEQ ID NO: 41 - protein, 16 aa, synthetic construct
LISSDGWTFYADSVKG

SEQ ID NO: 42 - protein, 15 aa, synthetic construct
SVLVLRTLNPKAWHH

SEQ ID NO: 43 - protein, 6 aa, synthetic construct
GDITRR

SEQ ID NO: 44 - protein, 5 aa, synthetic construct
SEDGI

SEQ ID NO: 45 - protein, 17 aa, synthetic construct
SILNRVGCRQLSYEYIY

SEQ ID NO: 46 - protein, 7 aa, synthetic construct
DNTFSRG

SEQ ID NO: 47 - protein, 5 aa, synthetic construct
SSDGW

SEQ ID NO: 48 - protein, 15 aa, synthetic construct
SFLVLRALNPKAWHY

SEQ ID NO: 49 - protein, 7 aa, synthetic construct
GFPSSRG

SEQ ID NO: 50 - protein, 5 aa, synthetic construct
NSDGR

SEQ ID NO: 51 - protein, 15 aa, synthetic construct
SLLYLMTVNAAAYPY

SEQ ID NO: 52 - protein, 7 aa, synthetic construct
GYTSNRY

SEQ ID NO: 53 - protein, 6 aa, synthetic construct
WTDGGR

SEQ ID NO: 54 - protein, 15 aa, synthetic construct
SSLLLQTINPRAWPY

SEQ ID NO: 55 - protein, 7 aa, synthetic construct
GDTFSRG

SEQ ID NO: 56 - protein, 5 aa, synthetic construct
SSDGW

SEQ ID NO: 57 - protein, 15 aa, synthetic construct
SVLVLRTLNPKAWHH

SEQ ID NO: 58 - protein, 99 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 59 - protein, 98 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 60 - protein, 98 aa, synthetic construct
QVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAK

SEQ ID NO: 61 - protein, 11 aa, synthetic construct
WGQGTMVTVSS

SEQ ID NO: 62 - protein, 124 aa, synthetic construct
EVQLVESGGGLVQPGRSLRLSCAASGDITRRCMHWVRQAPGKGLEWVSGISEDGITGYADSVKGRFTISRDNAKNSLYLQMNSLRAEDTALYYCAASILNRVGCRQLSYEYIYWGQGTMVTVSS

SEQ ID NO: 63 - protein, 123 aa, synthetic construct
EVQLVESGGGLVQPGGSLRLSCAASGFTFSRGSMSWVRQAPGKGLEWVAIISSDGWTGYADSVKGRFTISRDNAKNSLYLQMNSLRAEDTAVYYCARSFLVLRALNPKAWHYWGQGTMVTVSS

SEQ ID NO: 64 - protein, 123 aa, synthetic construct
EVQLVESGGGLVQPGGSLRLSCAASGFTFSRGTMAWVRQAPGKGLEWVAFINSDGRTSYAESVKGRFTISRDNAKNSLYLQMNSLRAEDTAVYYCARSLLYLMTVNAAAYPYWGQGTMVTVSS

SEQ ID NO: 65 - protein, 124 aa, synthetic construct
QVQLVESGGGVVQPGGSLRLSCAASGYTSNRYSMHWVRQAPGKGLEWVAFIWTDGGRTYYADSVKGRFTISRDNSKNTLYLQMNSLRAEDTAVYYCAASSLLLQTINPRAWPYWGQGTMVTVSS

SEQ ID NO: 66 - protein, 123 aa, synthetic construct
QVQLVESGGGVVQPGGSLRLSCAASGFTFSRGSWGWVRQAPGKGLEWVALISSDGWTFYADSVKGRFTISRDNSKNTLYLQMNSLRAEDTAVYYCAKSVLVLRTLNPKAWHHWGQGTMVTVSS

SEQ ID NO: 67 - protein, 124 aa, synthetic construct
EVQLVESGGGLVQPGRSLRLSCAASGDITRRCMGWFRQAPGKEREGVAGISEDGITIYADSVKGRFTISKDNAKNSLYLQMNSLRAEDTALYSCAASILNRVGCRQLSYEYIYRGQGTMVTVSS

SEQ ID NO: 68 - protein, 124 aa, synthetic construct
EVQLVESGGGLVQPGRSLRLSCAASGDITRRCMGWFRQAPGKEREGVAGISEDGITGYADSVKGRFTISRDNAKNSLYLQMNSLRAEDTALYYCAASILNRVGCRQLSYEYIYRGQGTMVTVSS

SEQ ID NO: 69 - protein, 124 aa, synthetic construct
EVQLVESGGGLVQPGRSLRLSCAASGDITRRCMGWFRQAPGKEREGVAGISEDGITGYADSVKGRFTISRDNAKNSLYLQMNSLRAEDTALYSCAASILNRVGCRQLSYEYIYRGQGTMVTVSS

SEQ ID NO: 70 - protein, 124 aa, synthetic construct
EVQLVESGGGLVQPGRSLRLSCAASGDITRRCMGWFRQAPGKEREGVAGISEDGITGYADSVKGRFTISKDNAKNSLYLQMNSLRAEDTALYSCAASILNRVGCRQLSYEYIYRGQGTMVTVSS

SEQ ID NO: 71 - protein, 124 aa, synthetic construct
EVQLVESGGGLVQPGRSLRLSCAASGDITRRCMGWFRQAPGKEREGVAGISEDGITGYADSVKGRFTISKDNAKNSLYLQMNSLRAEDTALYSCAASILNRVGCRQLSYEYIYRGQGTQVTVSS

SEQ ID NO: 72 - protein, 123 aa, synthetic construct
EVQLVESGGGLVQPGGSLRLSCAASGFTFSRGSMSWFRQAPGKEREGVAIISSDGWTGYADSVKGRFTISRDNAKNSLYLQMNSLRAEDTAVYYCAASFLVLRALNPKAWHYWGQGTMVTVSS

SEQ ID NO: 73 - protein, 123 aa, synthetic construct
EVQLVESGGGLVQPGGSLRLSCAASGFTFSRGSMSWFRQAPGKEREGVAIISSDGWTGYADSVKGRFTISQDNAKNSLYLQMNSLRAEDTAVYYCAASFLVLRALNPKAWHYWGQGTMVTVSS

SEQ ID NO: 74 - protein, 123 aa, synthetic construct
EVQLVESGGGLVQPGGSLRLSCAASGNTFSRGSMSWFRQAPGKEREGVAIISSDGWTGYADSVKGRFTISQDNAKNSLYLQMNSLRAEDTAVYYCAASFLVLRALNPKAWHYWGQGTMVTVSS

SEQ ID NO: 75 - protein, 123 aa, synthetic construct
DVQLVESGGGLVQPGGSLRLSCAASDNTFSRGSMSWFRQAPGKEREGVAIISSDGWTGYADSVKGRFTISQDNAKNSLYLQMNSLRAEDTAVYYCAASFLVLRALNPKAWHYWGQGTMVTVSS

SEQ ID NO: 76 - protein, 123 aa, synthetic construct
DVQLQASGGGLVQPGGSLRLSCAASDNTFSRGSMSWFRQAPGKEREGVAIISSDGWTGYADSVKGRFTISQDNAKNSLYLQMNSLRAEDTAVYYCAASFLVLRALNPKAWHYWGQGTMVTVSS

SEQ ID NO: 77 - protein, 123 aa, synthetic construct
DVQLVESGGGLVQPGGSLRLSCAASDNTFSRGSMSWFRQAPGKEREGVAIISSDGWTGYADSVKGRFTISQDNAKNSLYLQMNSLRAEDTAMYYCAASFLVLRALNPKAWHYWGQGTQVTVSS

SEQ ID NO: 78 - protein, 123 aa, synthetic construct
EVQLVESGGGLVQPGGSLRLSCAASGFTFSRGTMAWFRQAPGKEREGVAFINSDGRTSYAESVKGRFTISRDNAKNSLYLQMNSLRAEDTAVYYCAASLLYLMTVNAAAYPYWGQGTMVTVSS

SEQ ID NO: 79 - protein, 123 aa, synthetic construct
EVQLVESGGGLVQPGGSLRLSCAASGFPSSRGTMAWFRQAPGKEREGVAFINSDGRTSYAESVKGRFTISRDNAKNSLYLQMNSLRAEDTAVYYCAASLLYLMTVNAAAYPYWGQGTMVTVSS

SEQ ID NO: 80 - protein, 123 aa, synthetic construct
DVQLVESGGGLVQPGGSLRLSCAASGFPSSRGTMAWFRQAPGKEREGVAFINSDGRTSYAESVKGRFTISRDNAKNSLYLQMNSLRAEDTAVYYCAASLLYLMTVNAAAYPYWGQGTMVTVSS

SEQ ID NO: 81 - protein, 123 aa, synthetic construct
DVQLVESGGGLVQPGGSLRLSCAASGFPSSRGTMAWFRQAPGKEREGVAFINSDGRTSYAESVKGRFTISRDNALSSLYLQMNSLRAEDTAVYYCAASLLYLMTVNAAAYPYWGQGTMVTVSS

SEQ ID NO: 82 - protein, 123 aa, synthetic construct
DVQLVESGGGLVQPGGSLRLSCAASGFPSSRGTMAWFRQAPGKEREGVAFINSDGRTSYAESVKGRFTISRDNALSSLWLQMNSLRAEDTAVYYCAASLLYLMTVNAAAYPYWGQGTMVTVSS

SEQ ID NO: 83 - protein, 123 aa, synthetic construct
DVQLQASGGGLVQPGGSLRLSCAASGFPSSRGTMAWFRQAPGKEREGVAFINSDGRTSYAESVKGRFTISRDNALSSLYLQMNSLRAEDTAVYYCAASLLYLMTVNAAAYPYWGQGTMVTVSS

SEQ ID NO: 84 - protein, 123 aa, synthetic construct
DVQLVESGGGLVQPGGSLRLSCAASGFPSSRGTMAWFRQAPGKEREGVAFINSDGRTSYAESVKGRFTISRDNALSSLYLQMNSLRAEDTAMYYCAASLLYLMTVNAAAYPYWGQGTQVTVSS

SEQ ID NO: 85 - protein, 124 aa, synthetic construct
EVQLVESGGGVVQPGGSLRLSCAASGYTSNRYSMAWFRQAPGKEREVVAFIWTDGGRTYYADSVKGRFTISRDNSKNTLYLQMNSLRAEDTAVYYCAASSLLLQTINPRAWPYWGQGTMVTVSS

SEQ ID NO: 86 - protein, 124 aa, synthetic construct
EVQLVESGGGVVQPGGSLRLSCAASGYTSNRYSMAWFRQAPGKEREVVAFIWTDGGRTYYADSVKGRFTISHDNSKNTLYLQMNSLRAEDTAVYYCAASSLLLQTINPRAWPYWGQGTMVTVSS

SEQ ID NO: 87 - protein, 124 aa, synthetic construct
DVQLVESGGGVVQPGGSLRLSCAASGYTSNRYSMAWFRQAPGKEREVVAFIWTDGGRTYYADSVKGRFTISHDNSKNTLYLQMNSLRAEDTAVYYCAASSLLLQTINPRAWPYWGQGTMVTVSS

SEQ ID NO: 88 - protein, 124 aa, synthetic construct
DVQLVESGGGVVQPGGSLRLSCAASGYTSNRYSMAWFRQAPGKEREVVAFIWTDGGRTYYADSVKGRFTISHDNAKNTLYLQMNSLRAEDTAVYYCAASSLLLQTINPRAWPYWGQGTMVTVSS

SEQ ID NO: 89 - protein, 124 aa, synthetic construct
DVQLVESGGGVVQPGGSLRLSCAASGYTSNRYSMAWFRQAPGKEREVVAFIWTDGGRTYYADSVKGRLTISHDNAKNTLYLQMNSLRAEDTAVYYCAASSLLLQTINPRAWPYWGQGTMVTVSS

SEQ ID NO: 90 - protein, 124 aa, synthetic construct
DVQLQASGGGVVQPGGSLRLSCAASGYTSNRYSMAWFRQAPGKEREVVAFIWTDGGRTYYADSVKGRFTISHDNSKNTLYLQMNSLRAEDTAVYYCAASSLLLQTINPRAWPYWGQGTMVTVSS

SEQ ID NO: 91 - protein, 124 aa, synthetic construct
DVQLVESGGGVVQPGGSLRLSCAASGYTSNRYSMAWFRQAPGKEREVVAFIWTDGGRTYYADSVKGRFTISHDNSKNTLYLQMNSLRAEDTAMYYCAASSLLLQTINPRAWPYWGQGTQVTVSS

SEQ ID NO: 92 - protein, 123 aa, synthetic construct
EVQLVESGGGVVQPGGSLRLSCAASGFTFSRGSWGWFRQAPGKEREAVALISSDGWTFYADSVKGRFTISRDNSKNTLYLQMNSLRAEDTAVYYCAASVLVLRTLNPKAWHHWGQGTMVTVSS

SEQ ID NO: 93 - protein, 123 aa, synthetic construct
EVQLVESGGGVVQPGGSLRLSCAASGFTFSRGSWGWFRQAPGKEREAVALISSDGWTFYADSVKGRFTISQDNSKNTLYLQMNSLRAEDTAVYYCAASVLVLRTLNPKAWHHWGQGTMVTVSS

SEQ ID NO: 94 - protein, 123 aa, synthetic construct
EVQLVESGGGVVQPGGSLRLSCAASGDTFSRGSWGWFRQAPGKEREAVALISSDGWTFYADSVKGRFTISQDNSKNTLYLQMNSLRAEDTAVYYCAASVLVLRTLNPKAWHHWGQGTMVTVSS

SEQ ID NO: 95 - protein, 123 aa, synthetic construct
EVQLVESGGGVVQPGGSLRLSCAASGDTFSRGSWGWFRQAPGKEREAVALISSDGWTFYADSVKGRFTISQDNTKNTLYLQMNSLRAEDTAVYYCAASVLVLRTLNPKAWHHWGQGTMVTVSS

SEQ ID NO: 96 - protein, 123 aa, synthetic construct
EVQLVESGGGVVQPGGSLRLSCAASGDTFSRGSWGWFRQAPGKEREAVALISSDGWTFYADSVKGRFTISQDNSKNTLYLQMNSLRAEDTAMYYCAASVLVLRTLNPKAWHHWGQGTQVTVSS

SEQ ID NO: 97 - protein, 123 aa, synthetic construct
EVQLVESGGGVVQPGGSLRLSCAASGDTFSRGSWGWFRQRPGKEREAVALISSDGWTFYADSVKGRFTISQDNSKNTLYLQMNSLRAEDTAMYYCAASVLVLRTLNPKAWHHWGQGTQVTVSS

SEQ ID NO: 98 - protein, 123 aa, synthetic construct
EVQLQASGGGVVQPGGSLRLSCAASGDTFSRGSWGWFRQAPGKEREAVALISSDGWTFYADSVKGRFTISQDNSKNTLYLQMNSLRAEDTAVYYCAASVLVLRTLNPKAWHHWGQGTMVTVSS